(19) United States
(12) Patent Application Publication
(10) Pub. No.: US 2012/0107396 A1
(43) Pub. Date: May 3, 2012

(54) Compositions For The Treatment of Central Nervous System Disorders Including Depression Employing Novel Drug Combination Therapy To Reduce Suicidality In Patients

(76) Inventor: Arifulla Khan, Mercer Island, WA (US)

(21) Appl. No.: 13/342,994

(22) Filed: Jan. 4, 2012

Related U.S. Application Data
(63) Continuation of application No. 13/053,106, filed on Mar. 21, 2011, now abandoned.
(60) Provisional application No. 61/341,072, filed on Mar. 25, 2010.

(51) Int. Cl. A61K 9/20; A61K 33/00; A61P 25/18; A61P 25/28; A61P 25/22; A61P 3/00; A61P 3/04; A61P 25/30; A61P 25/36; A61P 25/08; A61P 25/16; A61P 15/00; A61P 25/00; A61P 25/24; A61K 9/00

(52) U.S. Cl. 424/464; 424/400; 424/600

(57) ABSTRACT

Described herein are novel methods and formulations for reducing suicidality in human subjects. Such formulations and methods are a combination of lithium and one or more other CNS therapeutic agents such as anti-depressant, mood-stabilizing, anxiolytic, anticonvulsant, antipsychotic, anti-addictive, and appetite suppressant drugs.

### RELATED APPLICATIONS

This application claims priority benefit of U.S. Provisional patent application Ser. No. 61/341,072, filed Mar. 25, 2010, which is incorporated herein by reference in its entirety.

### TECHNICAL FIELD

The present invention relates to methods and compositions for reducing suicidality in individuals at risk for suicide.

### BACKGROUND OF THE INVENTION

Over one million people commit suicide every year; this represents a global mortality rate of 16 per 100,000, or one suicide every 40 seconds (World Health Organization, 2011). It is the tenth leading cause of death in the United States (National Institutes of Mental Health, 2007). Although the evaluation and management of depressed and suicidal patients is one of the most common and difficult encounters in psychiatry, decisions regarding treatment of suicidality are generally not based on research data. The conventional wisdom for the treatment of suicidality is to diagnose a patient's psychiatric illness and treat that condition. It is assumed that this intervention will reduce suicide risk.

Humans are vulnerable to a wide variety of central nervous system (CNS) disorders including various psychopathological conditions which generally have complex and poorly understood neurophysiological causes. CNS disorders of particular concern within the present invention include depression, mood spectrum disorders, anxiety disorders, addictive disorders, and appetite disorders among others, which have vast medical impacts and costs worldwide. These and other CNS disorders are variably treated using different classes of drugs, including anti-depressant, mood-stabilizing, anxiolytic, anticonvulsant, antipsychotic, anti-addictive, and appetite suppressant drugs. Individuals with CNS disorders are associated with an increased risk of suicide, yet suicide risk is generally not specifically treated.

CNS disorders classified as mental health or psychiatric diseases generally provide a unique set of complications for clinicians, patients, and caregivers. Major depression, for instance, is a monumental health problem which poses tremendous financial burdens on society both in terms of health care costs and lost productivity of individuals suffering from depression. Such individuals are often unable to function in everyday life situations, in part because of feelings of extreme hopelessness and worthlessness.

The American Psychiatric Association recognizes several types of clinical depression, including mild depression (dysthymia), major depression, and bipolar disorder (manic-depression). Clinical depression, including both bipolar disorders and major depression disorders, affects an estimated 8 to 12% of the population worldwide (Andrade, 2003). In the U.S. alone, the adult population suffering from depression in any given year is close to 19 million people. (Kessler, 2005). The lifetime risk for a major depressive episode varies by country and is approximately 16.9% in the U.S. regardless of gender. (Andrade, 2003). When one episode of depression is experienced, there is a 50% likelihood of recurrent episodes. When a second episode of depression occurs, there is an 80-90% likelihood of recurrent episodes and 75% of depressive disorders are recurrent.

Depressive disorders are associated with significantly elevated incidence and severity of suicidality, including increased suicidal ideation and higher frequencies of suicide attempts and suicide completions. Approximately 20% of individuals suffering from a depressive disorder will attempt suicide, and 6% will actually complete suicide. 75% of all subjects who commit suicide have a depressive disorder. (Gotlib and Hammen, 2002)

Major depression is defined by a constellation of chronic symptoms that include sleep problems, appetite problems, anhedonia or lack of energy, feelings of worthlessness or hopelessness, difficulty concentrating, and suicidal thoughts. About 6.7% of the U.S. population suffers from major depression (Kessler, 2005) from different reports between approximately 10-25 percent of these patients commit suicide during the course of their disease.

Bipolar disorder is a distinct depressive condition prevalent across all cultures and age groups. About 10% of people with depression also experience episodes of mania and therefore fit the criteria for bipolar disorder. Bipolar depressive episodes usually last longer and have a greater likelihood of psychotic features compared to other depressive illnesses. In addition, bipolar disorders are associated with an extreme elevated risk of suicide, at least comparable to suicide rates observed in patients with major depression and psychotic disorders.

Anxiety disorders are characterized by disturbances of mood, thinking, and behavior, and often include physical symptoms such as tachycardia, increased respiration, sweating and tremor. In severe forms anxiety is significantly disabling leading to major health care costs and related financial losses. Anxiety disorders are among the most common mental disorders and can greatly limit quality of life. There are a wide variety of anxiety disorders, including panic disorders, generalized anxiety disorder, phobias, obsessive-compulsive disorder, and post-traumatic stress disorder, among others. As with the other CNS disorders described herein, anxiety disorders are often associated with elevated suicidality.

Appetite disorders include, for example, bulimia nervosa, anorexia nervosa, obesity, and eating disorders not otherwise specified (NOS). These and other recognized appetite and eating disorders also account for extraordinary morbidity, medical costs and other societal losses, and like other CNS disorders described herein are associated with elevated suicidality.

Addictive disorders, including alcohol and drug addictive disorders, are also very common and have a major impact on quality of life and health care and other associated costs. As with other CNS disorders described herein, addictive disorders are usually accompanied by elevated incidence and severity of suicidality among patients.

Other CNS conditions that impose major societal costs and have high morbidity, including elevated suicidality, include, psychotic disorders, Alzheimer's disease, Parkinson's disease, epilepsy, seizure disorders, tic disorders, attention deficit/hyperactivity disorders, learning disorders, impulse control disorders, oppositional defiant disorders, conduct disorders, abnormal sexual behaviors, schizoid behaviors, somatization, sleep disorders, autism spectrum disorders, and other mood disorders and mental illnesses. Various CNS conditions amenable to treatment using the methods and compositions of the invention include any of a range of conditions characterized as “personality disorders”, including but not limited to, Borderline Personality disorder, Obsessive Compulsive Disorder (OCD), Anti-social Personality Disorder, and others according to DSM-IV-TR classification (Diagnostic and Statistical Manual of Mental Disorders, Fourth Edition, Text Revision, 2000).

Effective medical treatments for the different CNS disorders described herein vary, however they often overlap and frequently involve similar modalities and tools of medication and psychotherapy for successful intervention. Because different CNS conditions may involve common, or related, neurophysiological pathways, mechanisms and agents, neuropharmacological interventions to treat diverse CNS conditions are often directed toward the same molecular or biochemical target(s). For example, many CNS disorders, including depressive disorders, anxiety disorders, and addictive disorders, are attributed at least in part to defects or imbalances in biochemical signaling in the CNS. Aberrant expression or physiology of one or more monoamine neurotransmitter receptors, for example, serotonin, norepinephrine, and/or dopamine receptors for these neurotransmitters, may mediate abnormal CNS signal transduction leading to mood disorders and other abnormal neurophysiological conditions.

In the case of depressive disorders and a variety of other CNS disorders contemplated herein, effective medical treatment often includes pharmacological intervention employing one or more “anti-depressant” drugs. Among the anti-depressant drugs employed for treatment of CNS disorders, there are three major classes of drugs in long-term, widespread use. These conventional anti-depressant drug classes include tricyclic anti-depressants (TCAs), selective serotonin reuptake inhibitors (SSRIs) and monoamine oxidase inhibitors (MAOIs). Other classes and species of drug agents that have been developed and used, with variable success, to treat depression and other CNS disorders include mood-stabilizing, anxiolytic, anticonvulsant, antipsychotic, anti-addictive, and appetite suppressant drugs. The efficacy and safety profiles of these different classes of drugs are generally well-known in the art.

A common feature of anti-depressants and other types of drugs for treating CNS conditions is a typically substantial adverse side effect profile. Among the most significant side effects of anti-depressants and other CNS therapeutic drugs is a risk of dependency among subjects taking these drugs, particularly in cases of long-term treatment. In addition to dependency risks, adverse side effects that may attend the use of anti-depressants and other CNS drugs may include, for example, sedation/drowsiness, euphoria, dizziness, headache, sleep disturbance, appetite changes, weight gain, dry mouth, sweating, rash, sexual dysfunction, loss of energy, fatigue, vision disturbances, adverse interactions with other drugs and/or alcohol, toxicity, and other side effects.

A paradoxical finding for anti-depressant drugs and other classes of CNS drugs is that, while the drugs are generally effective to treat symptoms and conditions of depression or another targeted CNS disorder, these drugs are generally not effective for reducing suicidality in patients. Thus, for example, Khan and coworkers evaluated suicidality in a large assemblage of patients treated with anti-depressants or placebo in clinical trials using U.S. Food and Drug Administration (FDA) records. Among psychiatric patients who participated in large-scale, placebo-controlled clinical trials, the FDA data indicated that suicidality was not reduced in anti-depressant-treated subjects (i.e., as compared to control subjects who received only placebo). Contrary to expectations, Khan and his colleagues observed approximately the same levels of suicidality in placebo control groups compared to anti-depressant-treated groups. These findings were consistent across a variety of psychiatric disorders (Khan, et al, 2000, Khan, et al, 2001, Khan, et al, 2001, and Khan, et al, 2002).

Further complicating the role of CNS therapeutics in relation to suicide risks among patients suffering from CNS disorders, the FDA issued a black box warning in 2004 reflecting a concern that certain anti-depressant drugs might actually contribute to increased suicidality in depressed patients taking the drugs, particularly children and adolescent subjects. The significance of the FDA's findings in this context, suggesting a possible causal link between certain anti-depressants and elevated suicide risks is unclear, because the underlying studies were poorly designed.

Suicide attributed to CNS disorders is a major cause of death, and a leading cause of death in young people, worldwide. In the United States alone, suicide claims the lives of roughly 33,000 people each year (CDC). Between 600,000 and 800,000 Americans attempt suicide annually. The overall financial toll of suicidality in the US (as a sequel of depressive disorders and all other causes) is on the scale of billions of dollars, which many consider overshadowed by the anguish and suffering suicide brings to hundreds of thousands of families each year.

The traditional management of suicidal patients has been hospitalization, psychiatric diagnosis, and treatment involving medication and/or psychotherapy. The prospect of a pharmacological treatment for suicidality among patients suffering from CNS disorders is a relatively new concept, and attempts at drug development toward this objective have met with little success. In one recent study, Meltzer and colleagues reported a potential role of clozapine in reducing suicide risk (Meltzer, et al, 2003). This study involved a large clinical trial conducted over several years, which assessed the use of clozapine compared to olanzapine. According to Meltzer's report, clozapine reduced suicide risk among study subjects (based on frequency of completed suicides and suicide attempts in two groups of patients with schizophrenia). However, considering the design of Meltzer's study and the data observed, the overall efficacy of clozapine for reducing suicide risk in patients suffering from CNS disorders remains uncertain.

Another drug that has been studied for its potential effects in reducing suicidality among CNS disorder patients is lithium, which has been in long-term use for treating bipolar disorders and certain other CNS conditions. Lithium is a basic element initially reported as a mood stabilizer by John Cade in 1949 (Cade, 1949). Despite its long-term use as a mood stabilizer, lithium has remained poorly understood for its potential utilities for treating other CNS disorders and related symptoms, including suicidality in any context, and particularly in the context of patients presenting with a CNS disorder as described herein.

Potential anti-suicidal properties of lithium were not reported for nearly a quarter of a century after the discovery that lithium possesses mood stabilizing properties. Perhaps the earliest reports of a prospective role of lithium in reducing suicidality was made by Dr. Prien and coworkers in 1974, who proposed that long-term treatment with lithium may reduce suicide risk (Prien, et al, 1974). A later study which evaluated suicidal indices among 362 subjects over a 7 year period (representing 3911 subject exposure years) concluded that suicide risk was reduced by 77% in participating patients presenting with mood disorders and schizoaffective disorder (Nilsson, 1999). In a subsequent study, Baldessarini and colleagues reviewed results of 33 studies and compared rates of suicide between study patients during periods of treatment with lithium compared to off-treatment periods when the patients did not receive lithium therapy (Baldessarini, et al, 2001). The authors reported that overall rates of suicidal acts (suicide attempts and completed suicides) were 3-fold greater off-treatment than on lithium treatment.

From these and other studies it remained unclear whether the reported anti-suicidal effects of lithium were related to its mood stabilizing effect, or to a separate property of lithium. In this context, a later study of U.S. patients with bipolar disorder reported that patients treated with lithium had a 2.7-fold lower suicide risk than patients treated with a different mood-stabilizer drug, depakote (Goodwin, et al, 2003). Additional evidence that lithium may possess anti-suicidal properties in certain contexts was presented in a more recent meta-analysis by Cipriani and coworkers (Cipriani, 2005). In this study, lithium not only was reported to reduce the incidence of completed suicides, but also to lower the incidence of self-harm acts overall. In a more recent meta-analysis lithium was reported to reduce suicide profiles among patients with major depressive disorder (Guzetta, et al., 2007). Other, more anecdotal evidence exists which further suggest a potential role for lithium in reducing suicidality among human subjects, including a report that in certain areas in Texas, high lithium levels in drinking water were associated with lower suicide rates (along with reduced crimes and arrests) (Scruazer and Shrestha, 1990). More recently, a group of Japanese investigators (Ohgami, et al., 2009) reported a significant correlation between suicide rates and drinking water levels of lithium across eighteen municipalities. The areas with the highest lithium levels (0.05 mg/L of drinking water) reportedly correlated with the lowest rates of suicide.

In summary, despite the high prevalence of suicidality among patients suffering from CNS disorders and the extraordinary financial and societal costs of suicide, there has yet to be developed any effective, approved treatments for preventing or treating suicide. While elevated suicide risk correlates strongly with many CNS disorders, including depression, anxiety, addictive disorders, and others, not a single drug among the vast armamentarium of CNS therapeutic drugs has shown any definitive, positive effects for preventing or treating suicide (Jamison et al., 1999, Khan, et al, 2000, Khan, et al, 2001, Khan, et al, 2001, and Khan, et al, 2002). In a recent study by Khan and coworkers analyzing suicides and suicide attempts among 46,575 patients, no reduction of suicidality was observed following treatment with any of 9 anti-depressants, 3 antipsychotics, and 7 anxiolytics, compared to placebo controls (Khan et al., 2006). Similar findings were reported by Gunell and coworkers (1994), and by the American Psychiatric Association (APA) Work Group on Suicidal Behaviors (2003) (based on a comprehensive literature review of over 34,000 publications). As characterized by Jamison and coworkers, “proof remains elusive that any medical intervention, including the recent development of safer anti-depressants that are not lethal on acute overdose, has produced a measurable impact on suicide rates in the general population.” Even the reports relating to a prospective role of lithium for treating or preventing suicide fail to substantiate an accepted indication for the use of lithium to treat or prevent suicide. While reports on lithium may suggest this utility, the mechanisms of action of lithium are not known, and there has never been a direct measure of lithium's anti-suicidal effects assessed using a well-designed prospective clinical trial. In fact, suicidal patients are routinely excluded from psychopharmacology trials for perceived excessive risk. This lack of clarity has further fuelled concerns about the potential suicide-inducing capacity of antidepressants and several other classes of drugs.

There is therefore an unmet need in the art for the identification of methods and compositions for treating and preventing suicidality in humans.

### SUMMARY OF THE EXEMPLARY EMBODIMENTS OF THE INVENTION

The instant invention provides novel methods and compositions for preventing or treating suicidality in human subjects. Additionally provided herein are methods and compositions for preventing or treating suicidality in individuals suffering from central nervous system (CNS) disorders. Further provided are methods and compositions for preventing or treating suicidality and CNS disorders in individuals suffering from such disorders. The methods of the invention employ coordinate administration of a CNS therapeutic agent in an amount effective to treat a CNS disorder, along with administration of a lithium agent (e.g., a lithium salt) in an amount effective to reduce suicidality in the subject.

Within additional aspects of the invention, combinatorial formulations are provided which employ an active CNS therapeutic agent co-formulated with a lithium agent to treat a CNS disorder in the subject and simultaneously reduce suicidality in the subject.

The combinatorial formulations and coordinate treatment methods of the invention are effective to modulate, alleviate, treat, or prevent one or more symptom(s) of a targeted CNS disorder in mammalian subject while reducing suicidality in treated patients suffering from the subject CNS disorder. Exemplary combinatorial formulations and coordinate treatment methods in this context employ an active anti-depressant or other CNS therapeutic drug in combination with a lithium salt to treat depression or anxiety and simultaneously reduce suicidality associated with depression and anxiety in the subject.

In the coordinate administration methods of the invention, a CNS therapeutic drug is administered concurrently or sequentially in either order, with lithium to treat or prevent one or more symptoms of the targeted CNS disorder. When administered simultaneously, the CNS therapeutic agent and lithium may be combined in a single composition or combined dosage form, or administered at the same time in separate dosage forms.

Within various aspects of the invention targeted CNS disorders for treatment may be any of a variety of depressive disorders, mood disorders, psychoses, or other mental illnesses. In certain embodiments, the selected CNS disorder is a depressive disorder, which may be mild depression (dysthymia), a major depressive disorder, a depressive disorder not otherwise specified, seasonal affective disorder, bipolar disorder, cyclothymic disorder, neurotic depression, atypical (reactive) depression. Other, related mood disorders for treatment according to the methods and formulations of the invention include, for example, borderline personality disorder, mood disorders induced by alcohol or drugs, and any other mood disorder amenable to treatment using one or more CNS therapeutic drug(s) described herein.

In more detailed embodiments, the depressive disorder for coordinate treatment according to the invention is a major depressive disorder (single incidence or recurrent, unipolar depression). In alternate embodiments, the CNS disorder is a bipolar or manic depressive disorder, which in related embodiments will include bipolar I disorder or bipolar II disorder.

Within additional embodiments of the invention, the CNS disorder for coordinate treatment according to the invention is an anxiety disorder, which can include, for example, generalized anxiety disorder, anxiety disorder not otherwise specified, obsessive-compulsive disorder (OCD), stress disorders including post-traumatic stress disorder (PTSD), phobias, panic disorders, anxiety disorders induced by alcohol or drugs, and other anxiety disorders recognized in the art.

Within other embodiments of the invention, the CNS disorder for coordinate treatment according to the invention is an appetite or eating disorder, which can include, for example, bulimia nervosa, anorexia nervosa, obesity, and eating disorders not otherwise specified.

Within additional embodiments of the invention, the CNS disorder for coordinate treatment according to the invention is an addictive disorder, including for example any “Substance Related Disorder” according to DSM-IV-TR classification (American Psychiatric Association, 2000). Addictive disorders amenable to the treatment methods and compositions of the invention include, for example, alcohol-related addictive disorders, nicotine-related addictive disorders, amphetamine-related addictive disorders, cannabis-related addictive disorders, cocaine-related addictive disorders, hallucinogen use disorders, inhalant-related addictive disorders, opioid-related addictive disorders, and other addictive disorders known in the art.

Yet additional methods and compositions of the invention are effective for treating psychotic disorders, Alzheimer's disease, Parkinson's disease, epilepsy, seizure disorders, tic disorders, attention deficit/hyperactivity disorders, learning disorders, impulse control disorders, oppositional defiant disorders, conduct disorders, abnormal sexual behaviors, schizoid behaviors, somatization, sleep disorders, autism spectrum disorders, and other mood disorders and mental illnesses that differ from the depressive, anxiety, eating and addictive disorders set forth above. Exemplary psychotic disorders amenable to treatment with reduced suicidality employing the methods and compositions of the invention include, but are not limited to, schizophrenia, schizophreniform diseases, acute mania, schizoaffective disorders, depression with psychotic features, and other psychotic disorders known in the art and associated with increased risks of suicide.

Within the coordinate treatment methods and combinatorial formulations of the invention, a wide variety of CNS therapeutic agents may be selected, including drugs from each general class of anti-depressant, mood-stabilizing, anxiolytic, anticonvulsant, antipsychotic, anti-addictive, and appetite suppressant drugs.

In certain embodiments of the invention, the CNS therapeutic agent is an anti-depressant drug, which may include, for example, any species within the broad families of tri-cyclic anti-depressants (TCAs), specific monoamine reuptake inhibitors, selective serotonin reuptake inhibitors (SSRIs), selective norepinephrine reuptake inhibitors, serotonin-norepinephrine reuptake inhibitors, selective dopamine reuptake inhibitors, multiple monoamine reuptake inhibitors, monoamine oxidase inhibitors (MAOIs), and indeterminate (atypical) anti-depressants.

In more detailed embodiments of the invention, the methods and compositions employ an SSRI anti-depressant. Within exemplary embodiments, the SSRI is citalopram.

Other classes and species of drug agents have been developed and used, with variable success, to treat depression and other CNS disorders contemplated herein, and in general these other classes and species of drugs can be employed within the coordinate treatment methods and combinatorial formulations of the invention. Among the additional classes of drugs for treating CNS disorders, drugs having utility within the coordinate treatment methods and combinatorial compositions of the invention include, mood-stabilizing, anxiolytic, anticonvulsant, antipsychotic, anti-addictive, and appetite suppressant drugs. The efficacy and safety profiles of these different classes of drugs are generally well-known in the art.

Within various aspects of the invention, the CNS therapeutic agent and lithium may each be administered by any of a variety of delivery routes and modes, which may be the same or different for each agent. In alternate embodiments, the CNS therapeutic drug and/or lithium is administered by a mode of delivery selected from oral, buccal, nasal, aerosol, topical, transdermal, mucosal, or injectable. In exemplary embodiments, both the CNS therapeutic agent and lithium are administered orally. In other embodiments, one or both of the CNS drug and lithium is/are delivered in a sustained or extended release formulation, for example in a sustained release, oral, transdermal, or injectable (e.g., subcutaneous or intramuscular) formulation.

Within certain embodiments of the invention, the lithium is administered in an anti-suicidal effective dosage of from about 50 mg to about 2000 mg. In other embodiments, the lithium is administered in an anti-suicidal effective dosage of from about 100 mg to about 500 mg. In more detailed embodiments, the lithium is administered in an anti-suicidal effective dosage of about 500 mg or less, about 300 mg or less, or even lower.

In more detailed embodiments of the invention, the CNS drug and lithium are administered in a single, combined dosage form, for example admixed or co-formulated in a liquid or solid dosage form. In exemplary embodiments, the CNS drug and said lithium are admixed or co-formulated in an oral dosage form.

In other aspects of the invention, pharmaceutical compositions are provided for reducing suicidality in human subjects suffering from a central nervous system (CNS) disorders. The pharmaceutical compositions comprise a CNS therapeutic agent in an amount effective to treat the targeted CNS disorder and lithium in an amount effective to reduce suicidality in the subject. The pharmaceutical compositions may include the CNS therapeutic agent and lithium in separate dosage forms or admixed or co-formulated in a single, combined dosage form. In more detailed embodiments, the CNS drug and lithium are admixed or co-formulated in a liquid or solid dosage form, for example an oral dosage form. In other detailed embodiments, the pharmaceutical composition further comprises a package containing the CNS therapeutic agent and lithium, separate or combinatorially formulated in a single dosage form. The package may include a label or insert providing instructions for multiple uses of the composition for treating the subject CNS disorder and reducing suicidality in the subject.

In other embodiments the invention provides pharmaceutical kits for reducing suicidality in a human subject suffering from a central nervous system (CNS) disorder. The kits comprise a CNS therapeutic agent in an amount effective to treat the targeted CNS disorder, lithium in an amount effective to reduce suicidality in the subject, and a container means for containing the CNS therapeutic agent and said lithium for coordinate administration to the said subject. The container means can include a package bearing a label or insert that provides instructions for multiple uses of the kit contents to treat the CNS disorder and reducing suicidality in the subject. In more detailed embodiments, the CNS therapeutic agent and lithium are admixed or co-formulated in a single, combined dosage form, for example a liquid or solid oral dosage form. In alternate embodiments, the CNS therapeutic agent and lithium are contained in the kit in separate dosage forms for coordinate administration.

### DETAILED DESCRIPTION OF EXEMPLARY EMBODIMENTS

The present invention provides novel coordinate treatment methods and combined drug compositions, dosage forms, packages, and kiss for preventing or treating suicidality in human subject. Subjects amenable to treatment according to the invention include subjects suffering from any of a variety of specified central nervous system (CNS) disorders. Within the methods of the invention a CNS therapeutic agent is administered in an amount effective to treat a specified CNS disorder, and a lithium agent is coordinately administered with the CNS therapeutic agent in an anti-suicidal effective amount sufficient to prevent or reduce suicidality in the subject.

Within related aspects of the invention, combinatorial formulations are provided which employ an active CNS therapeutic agent co-formulated with a lithium agent to treat a CNS disorder in the subject and simultaneously reduce suicidality in the subject.

The combined formulations and coordinate treatment methods of the invention are effective to modulate, alleviate, treat or prevent one or more symptom(s) of a targeted CNS disorder in mammalian subject. Exemplary combinatorial formulations and coordinate treatment methods in this context employ an active anti-depressant or anxiolytic drug in combination with a lithium salt to treat depression or anxiety and simultaneously reduce suicidality associated with depression and anxiety in the subject.

Within the coordinate administration methods of the invention, the CNS therapeutic drug is administered concurrently or sequentially with lithium to treat or prevent one or more symptoms of the targeted CNS disorder. When administered simultaneously, the CNS therapeutic agent and lithium may be combined in a single composition or combined dosage form. Alternatively, the combinatorially effective CNS therapeutic and lithium two agents may be administered at the same time in separate dosage forms. When the coordinate administration is conducted simultaneously or sequentially, the CNS therapeutic agent and lithium agent may each exert biological activities and therapeutic effects over different time periods, although a distinguishing aspect of all coordinate treatment methods of the invention is that treated subjects exhibit both positive CNS therapeutic benefits, as well as reduced suicidality.

Dosing and therapeutic benefits of the CNS therapeutic drug coordinately administered with lithium will typically have similarly favorable therapeutic effects and comparable side effects as a therapeutic benefit and side effect profile achieved in control patients treated with the CNS therapeutic agent alone. However, in certain embodiments the dosage of the CNS therapeutic agent may be lowered and yet in combination with the lithium agent will still have comparable therapeutic benefits and similar side effects as a therapeutic benefit and side effect profile achieved in control patients treated with a higher dosage of the CNS therapeutic agent alone. Given that the primary focus of the methods and compositions of the invention is reduction of suicidality, in certain embodiments coordinate administration of the CNS therapeutic agent with lithium will only moderate or even insignificant clinical benefits for preventing or alleviating the CNS disorder against which the CNS therapeutic drug is directed (compared to control subjects treated only with the CNS therapeutic agent), while in all instances suicidality in coordinately treated subjects will be substantially reduced in comparison to control patients treated with the CNS therapeutic agent without the lithium agent.

Within more detailed embodiments of the invention, the combinatorial formulations and coordinate treatment methods of the invention achieve substantial therapeutic benefit in terms of a clinical reduction in incidence, rate, recurrence, or severity of a specified CNS disorder, such as a depressive or other mood disorder, anxiety disorder, or addictive disorder, while at the same time reducing suicidality in treated patients significantly below a suicidality incidence or severity observed in control patients treated with the CNS therapeutic agent alone. In related embodiments, the compositions and methods of the invention measurably alleviate or prevent one or more symptoms of a specified CNS disorder, for example one or more symptoms of a major depressive disorder or bipolar disorder, with a concomitant reduction of suicidality in treated versus control subjects. In some embodiments, the symptoms of a specified CNS disorder and/or suicidality may be altered at the same or different rates or to the same or different degrees, i.e. the clinical reduction in incidence, rate, recurrence or severity of a specified CNS disorder or suicidality may be affected independently of each other.

Administration of a coordinate treatment method or combinatorial drug composition of the invention to suitable subjects (e.g., qualified subjects participating in a validated, double blinded clinical trial for depression or anxiety) will yield a reduction in suicidality and/or one or more target symptom(s) associated with the selected CNS disorder by at least 5%, 10%, 20%, 30%, 50% or greater, up to a 75-90%, or 95% or greater, compared to placebo-treated or other suitable control subjects. Comparable levels of efficacy are contemplated for the entire range of CNS disorders described herein, including all contemplated neurological and psychiatric disorders, and related conditions and symptoms, for treatment or prevention using the compositions and methods of the invention. These values for efficacy may be determined by comparing accepted therapeutic indices or clinical values for particular test and control individuals over a course of treatment/study, or more typically by comparing accepted therapeutic indices or clinical values between test and control groups of individuals using standard human clinical trial design and implementation.

As used herein, the terms “prevention” and “preventing,” when referring to a CNS disorder or symptom, or a suicidal condition or indicator, refer to a reduction in the risk or likelihood that a mammalian subject will develop said disorder, symptom, condition, or indicator after treatment according to the invention, or will exhibit a recurrence of said disorder, symptom, condition, or indicator once a subject has been treated according to the invention and cured or restored to a normal state (e.g., placed in remission from a targeted CNS disorder and/or no longer exhibits symptoms of suicidality). As used herein, the terms “treatment” or “treating,” when referring to CNS disorders and suicidality refers to inhibiting or reducing the progression, nature, or severity of the subject condition or delaying the onset of the condition.

An “effective amount,” “therapeutic amount,” “therapeutically effective amount,” or “effective dose” of a CNS therapeutic agents and a lithium agents as used herein means an effective amount or dose of the active compound as described herein sufficient to elicit a desired pharmacological or therapeutic effect in a human subject. In the case of CNS therapeutic agents, these terms most often refer to a measurable, statistically significant reduction in an occurrence, frequency, or severity of one or more symptom(s) of a specified CNS disorder, including any combination of neurological and/or psychological symptoms, diseases, or conditions, associated with Dr caused by the targeted CNS disorder.

In certain embodiments, when a compound of the invention is administered to treat a CNS disorder, for example a depressive disorder, an effective amount of the CNS therapeutic agent will be an amount sufficient in vivo to delay or eliminate onset of one or more selected symptom(s) in an individual or between test and control groups of subjects, or to reduce or prevent the incidence, severity, duration, or recurrence of symptoms, or beneficially alter the nature of symptoms, in a test subject or group of test subjects compared to control subjects.

Therapeutic efficacy can alternatively be demonstrated by a decrease in the frequency or severity of symptoms associated with the treated condition or disorder, or by altering the nature, recurrence, or duration of symptoms associated with the treated condition or disorder. Therapeutic efficacy with the treated condition or disorder, or by altering the nature, recurrence, or duration of symptoms associated with the treated condition or disorder. In this context, “effective amounts,” “therapeutic amounts,” “therapeutically effective amounts,” and “effective doses” of CNS drugs and lithium agents within the invention can be readily determined by ordinarily skilled artisans following the teachings of this disclosure and employing tools and methods generally known in the art, often based on routine clinical or patient-specific factors.

With respect to clinical reduction or therapeutic remediation of suicidality in subjects coordinately treated with a lithium agent and CNS therapeutic drug according to the invention, these relative values can similarly be demonstrated by a decrease in the frequency or severity of one or more symptom(s) associated suicidality, including for example a decrease in the frequency or severity of suicidal ideation, suicidal impulsivity, suicidal communication, suicidal acts, suicide attempts, and/or completed suicide between test and control individuals or study groups. In exemplary embodiments, reduction of suicidality following coordinate treatment with a CNS therapeutic agent and lithium agent according to the invention may be determined as a decrease in a ratio of completed suicides to suicide attempts.

Efficacy of the coordinate treatment methods and combinatorial drug compositions of the invention will often be determined by the use of conventional patient surveys or clinical scales to measure clinical indices of CNS disorders and/or suicidality in subjects. The methods and compositions of the invention will yield a reduction in one or more scores or selected values generated from such surveys or scales completed by test subjects (indicating for example an incidence or severity of a selected CNS disorder or suicidal condition), by at least 5%, 10%, 20%, 30%, 50% or greater, up to a 75-90%, or 95% compared to correlative scores or values observed for control subjects treated with placebo or other suitable control treatment. More detailed data regarding efficacy of the methods and compositions of the invention can be determined using alternative clinical trial designs (for example comparing test subjects with positive control subjects treated with a different CNS therapeutic drug and/or mood stabilizing drug).

Useful patient surveys and clinical scales for comparative measurement of clinical indices of CNS disorders and/or suicidality in subjects treated using the methods and compositions of the invention can include any of a variety of widely used and well known surveys and clinical scales. Among these useful tools are the Hamilton Depression Rating Scale© (HDRS) (Hamilton, M., J. Neurol. Neurosurg. Psychiatr. 23:56-62, 1960; Hamilton, M., Br. J. Soc. Clin. Psychol. 6:278-296, 1967); Montgomery-Åsberg Depression Rating Scale© (HDRS) (Montgomery and Åsberg, 1979); Beck Scale for Suicide Ideation® (BSS) (Beck and Steer, 1991); Mini International Neuropsychiatric Interview© (MINI) (Sheehan et al., 1998; see pages 6-7 for Suicidality Assessment); Columbia-Suicide Severity Rating Scale© (C-SSRS) or Columbia Classification Algorithm of Suicide Assessment© (C CASA) (Posner, K, et al., 2007); Sheehan-Suicidality Tracking Scale© (S-SST) (Coric et al., 2009); Beck Hopelessness Scale© (BHS) (Beck, Steer, 1988); Geriatric Depression Scale (GDS) (Yesavage, J. A. et al., J. Psychiatr. Res. 17:37-49, 1983); and Clinical Global Impression scale (CGI) (Guy, W., ECDEU Assessment Manual for Psychopharmacology, DHEW Publication No. (ADM) 76-338, rev. 1976)). Any of these scales, alone or in combination, can be effectively employed to determine efficacy of the methods and compositions of the invention. Additionally, a variety of other scales and methods for assessing comparative CNS disorder symptoms or status, or suicidality indices or values, are widely used and well known in the art for use within the invention. Other exemplary scales for assessing efficacy of the invention with respect to other CNS disorders described herein include, for example, the HAM-A rating scale for anxiety (Hamilton, 1959); the HAM-D scale for depression (Hamilton, 1960); the Yale-Brown Obsessive Compulsive Scale (YBOCS) (Goodman et al., 1989); The Positive and Negative Syndrome Scale (PANSS) for schizophrenia (Kay et al., 1987); the YMRS rating scale for mania (Young et al., 1978); the Liebowitz Social Anxiety Scale (Liebowitz, 1987); and the Clinician-Administered Posttraumatic Stress Disorder Scale (CAPS) (Weathers et al., 1999). Comparably useful scales for determining efficacy or optimization of different aspects of the invention relating to additional CNS conditions described herein are well known in the art and readily employed for practicing alternate aspects of the invention. In certain embodiments of the invention, efficacy of the methods and compositions provided herein is determined by use of the MINI scale (The Mini International Neuropsychiatric Interview (e.g., version 6.0, January 2009)) a clinician-rated diagnostic assessment. In the exemplary protocol described in Example I below, the MINI test is administered at the first, screening visit and will be considered a source document. The MINI is administered by a psychiatrist, psychologist or master's level clinician with a minimum of 2 years experience in the diagnosis of mental illness. Also for use in demonstrating efficacy of the invention is the C-SSRS, which was developed by leading experts to assess suicide behavior and ideation. This test may be used to assess treatment benefit outcomes, safety outcomes, and clinical safety monitoring. The C-SSRS is a semi-structured-flexible format and may be completed by a physician, psychologist, or master's level clinician trained in the use of the scale. The S-SST scale can also be employed, alone or in combination with any of the other scales or like tools known in the art, for example as a clinician rated scale to determine efficacy and optimization (e.g., optimal dosage, frequency, duration of treatment). Likewise for the MADRS scale, a 10-item clinician rated scale to assess depression severity. Also useful in this context is the BHS, which in exemplary protocols as provided in Example I will be patient administered and scored by a clinician. The BHS measures the extent of negative attitudes about the future. It has a particular utility as an indirect indicator of suicidal risk in depressed examinees or individuals who have made suicide attempts. Yet another tool for use in exemplary clinical protocols is the BSS, a 21-item self-report instrument used to detect and measure the severity of suicidal ideation in adults. It measures a broad spectrum of attitudes and behaviors for assessing patient suicide risk, as well as reveals specific suicidal characteristics which require greater scrutiny. The CGI scale is also useful in these contexts, alone or in any combination with the other scales described herein. The CGI is a 7-point, clinician rated scale to rate illness severity, improvement and response to treatment. The severity of illness scale uses a range of responses from 1 to 7, with 1 being “normal” and 7 “amongst the most severely ill patients”.

The methods and compositions of the invention will yield a reduction in one or more scores or values generated from these clinical surveys (using any single scale or survey, or any combination of one or more of the surveys described above) by at least 5%, 10%, 20%, 30%, 50% or greater, up to a 75-90%, or 95% compared to correlative scores or values observed for control subjects treated with placebo or other suitable control treatment. For example, the Clinical Global Impression (CGI) scale is a 7-point, clinician rated scale to determine severity, improvement and response to treatment for selected CNS disorders, including depressive disorders. The CGI severity of illness scale uses a range of responses from 1 to 7, with 1 being “normal” and 7 “amongst the most severely ill patients” (Guy, 1976). A “responder” according to this measuring tool is defined as being “Much Improved” or “Very Much Improved”, having a CGI score of at least 2. Thus in one alternate expression of efficacy of the invention, a frequency of normal to moderately symptomatic CGI scores, for example scores of 1, 2, 3, or 4, will occur more often in subjects treated according to the invention, by at least 5%, 10%, 20%, 30%, 50% or greater, up to a 75-90%, or 95% compared to a frequency of the same normal to moderately symptomatic scores or values observed for control subjects completing the CGI following administration of placebo. Yet another exemplary expression of efficacy for the coordinate treatment methods and combinatorial compositions of the invention involves the HDRS, where lower numerical rating in the HDRS scale signifies less depression. Following treatment according to the invention, a frequency of low HDRS scores, for example scores of less than 2 at a post-baseline visit, will occur more often by at least 10%, 20%, 30%, 50% or greater, up to a 75-90%, or 95% compared to a frequency of the same low HD scores observed in control subjects.

In related embodiments of the invention, efficacy of the coordinate treatment methods and combinatorial formulations described herein for reducing suicidality may be determined by comparative frequencies of suicidal ideation, suicide attempts, completed suicide, and/or other indices of suicidality measured using one or more of the BSS, MINI, C-SSRS, and/or S-SST scales. One or more of the positive indices (e.g., values or scores) of suicidality determined using these scales will be observed less frequently in subjects treated according to the invention, by at least 5%, 10%, 20%, 30%, 50% or greater, up to a 75-90%, or 95% compared to a frequency of the same suicidal indices observed for control subjects completing the subject interview/scale after administration of placebo or other suitable control protocol.

For the purposes of describing the present invention, the following terms and definitions are provided by way of example. Additional terms and definitions for describing the present invention are provided by way of example elsewhere in the application.

Within various aspects of the invention targeted CNS disorders for treatment may include any of a variety of mood disorders, psychoses, or other mental illnesses. In certain embodiments, the selected CNS disorder is a depressive disorder, which may be selected from one or more of the following: mild depression (dysthymia), a major depressive disorder, a depressive disorder not otherwise specified (NOS), seasonal affective disorder, bipolar disorder, cyclothymic disorder, neurotic depression, atypical (reactive) depression, a mood disorder induced by alcohol or drugs, borderline personality disorder, and any other mood disorder amenable to treatment using one or more CNS therapeutic drug(s) described herein.

The combinatorial formulations and coordinate treatment methods of the invention achieve substantial clinical reduction of one or more symptom(s) associated with one or more of these depressive and/or and other mood disorder(s), while at the same time exhibiting substantial clinical reduction of reducing suicidality in treated patients significantly below a suicidality incidence or severity observed in control patients treated with the CNS therapeutic agent alone. By “substantial clinical reduction” is meant that one or more symptom(s) or indices of the targeted CNS disorder or suicidal condition will be observed less frequently in subjects treated according to the invention, by at least 5%, 10%, 20%, 30%, 50% or greater, up to a 75-90%, or 95% compared to a frequency of the same symptom(s) or indices observed for control subjects completing the subject interview/scale after administration of placebo or other suitable control.

CNS disorders manifest in a diverse array of symptoms amenable to treatment according to the methods and compositions of the invention. Most of the CNS disorders contemplated for treatment according to the invention are diagnosed and described according to criteria provided in widely used handbooks for psychiatry, for example in the Diagnostic and Statistical Manual of Mental Disorders 4th edition Text Revision (DSM-IV-TR) published by the American Psychiatric Association (APA; Washington, D.C., 2000).

In certain embodiments, the methods and compositions of the invention are directed towards the treatment of mood disorders. By “mood disorder” is meant a condition characterized by disruption of feeling tone or emotional state experienced by an individual for an extensive period of time. Mood disorders include major depression disorder (i.e., unipolar disorder), mania, dysphoria, bipolar disorder, dysthymia, cyclothymia and other related CNS disorders (See, e.g., Diagnostic and Statistical Manual of Mental Disorders-, Fourth Edition, Text Revision (DSM IV-TR)).

The APA recognizes several types of clinical depression, including mild depression (dysthymia), major depression, and bipolar disorder (manic-depression). Depressive disorders and conditions amenable to treatment according to the invention include, but are not limited to, any of the depressive disorders and conditions as described in the Diagnostic and Statistical Manual of Mental Disorders, Fourth Edition (Copyright 1994, American Psychiatric Association) and the Text Revision (Copyright 2000, American Psychiatric Association). These depressive disorders include major depressive disorder (unipolar depression), dysthymic disorder (chronic, mild depression), and bipolar disorder (manic-depression). Clinical depression refers to any form of depression that requires some form of treatment in order to alleviate it. Such clinical depression may persist for months, last for most of every day and seriously impairs the quality of life.

Certain embodiments of the invention are directed toward coordinate treatment of major depression and suicidality. The diagnostic criteria for major depression are well known to those skilled in the art, and comprise criteria set forth, for example, at DSM-IV 296.2 and 296.3. Major depression can be defined by a constellation of common, chronic symptoms that may include one or more of the following: sleep problems, appetite disorders (including loss of appetite, overeating, and obesity), loss of interest or pleasure in hobbies and activities that were previously enjoyed, anhedonia or lack of energy, fatigue, loss of libido, disruption of normal circadian rhythms of activity, body temperature, and endocrine functions, headache, restlessness, irritability, feelings of guilt, worthlessness, helplessness, or hopelessness, low self esteem, difficulty concentrating, overwhelming sadness or fear, despair, pessimistic worry, agitation, self-deprecation and/or suicidal thoughts or behaviors. Because depression tends to suppress emotion and reduce interest and pleasure, depressive disorders can also significantly reduce efficiency of work and ability of logical communication (Johnson et al., 1992).

As is recognized in the psychiatric art, depression may also manifest in a variety of other forms, including but not limited to, seasonal affective disorder, diurnal mood variations, or depression associated with menopause. Diagnosis criteria for dysthymia, as well as for seasonal affective disorder, diurnal mood variations and depression associated with menopause, are more fully explained in the Diagnostic and Statistical Manual of Mental Disorders, Fourth Edition, Text Revision (DSM IV TR). Depression with seasonal affective pattern or seasonal affective disorder (SAD) is also known as cabin fever, evening blues, and sun deprivation syndrome. The term “diurnal mood variation” refers to rhythmical daily changes in mood. It is described in the DSM-IV-TR as a form of atypical depression. Depression with diurnal mood variations (DMV) is often associated with increased appetite or weight gain, hypersomnia, leaden paralysis (unexplained tiredness and exhaustion), and a long-standing pattern of extreme sensitivity to perceived interpersonal rejection. This type of depression is more common in women and tends to be chronic, with the first depressive episode often occurring early in life (e.g. in high school).

In other embodiments the invention focuses on bipolar disorders, which typically involve major depressive episodes alternating with high-energy periods of rash behavior, poor judgment, and grand delusions. Persons with bipolar disorder often experience cycling of moods that usually swing from being overly elated or irritable (mania) to sad and hopeless (depression) and then back again, with periods of normal mood in between. Diagnosis of bipolar disorder is described in, e.g., DSM IV TR. Bipolar disorders include bipolar disorder I (mania with or without major depression) and bipolar disorder II (hypomania with major depression). Bipolar depressive episodes usually last longer, have a greater likelihood of psychotic features, and convey a greater risk of suicide than other forms of depression.

Bipolar Disorder is a recurrent disorder characterized by one or more manic episodes immediately before or after a major depressive episode, or may be characterized by one or more major depressive episodes accompanied by at least one hypomanic episode. Additionally, the symptoms of bipolar disorder often include clinically significant distress or impairment in social, occupational, or other important areas of functioning. In some cases of bipolar disorder, hypomanic episodes themselves do not cause impairment; however, the impairment may result from the major depressive episodes or from a chronic pattern of unpredictable mood episodes and fluctuating unreliable interpersonal and occupational functioning. Bipolar disorder may be misdiagnosed as depression resulting in inappropriate treatment that may worsen the disease progression and outcome.

Other mood disorders amenable to treatment employing the methods and compositions of the invention include dysthymic disorder with early or late onset and with or without atypical features; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics and other substances; schizoaffective disorder of the depressed type; adjustment disorder with depressed mood, and various impulse control and personality disorders.

In addition to the human screening methods described above, various animal models for depression are also well known to those of ordinary skill in the art. For instance, the efficacy of compounds and methods of the invention in the treatment of depression can be tested in a model of chronic mild stress induced anhedonia in rats. This model is based on the observation that chronic mild stress causes a gradual decrease in sensitivity to rewards, for example consumption of sucrose, and that this decrease is dose-dependently reversed by chronic treatment with anti-depressants. The method has previously been described and more information with respect to the test appears from Willner, Paul, Psychopharmacology, 1997, 134, 319 329.

Another animal model for anti-depressant activity is the forced swimming test (Nature 266, 730 732, 1977). In this test, animals are administered an agent preferably by the intraperitoneal route or by the oral route 30 or 60 minutes before the test. The animals are placed in a crystallizing dish filled with water and the time during which they remain immobile is clocked. The immobility time is then compared with that of the control group treated with distilled water. Imipramine 25 mg/kg can be used as the positive control. Anti-depressant compounds decrease the immobility time of the mice thus immersed. Yet another animal model for anti-depressant activity is the caudal suspension test on the mouse (Psychopharmacology, 85, 367 370, 1985). In this test, animals are preferably treated with the study compound by the intraperitoneal route or by the oral route 30 or 60 minutes before the test. The animals are then suspended by the tail and their immobility time is automatically recorded by a computer system. The immobility times are then compared with those of a control group treated with distilled water. Anti-depressant compounds decrease the immobility time of the mice.

Other CNS disorders amenable to treatment using the methods and compositions of the invention include anxiety disorders, including but not limited to any of the anxiety disorders as provided in the Diagnostic and Statistical Manual of Mental Disorders, Fourth Edition Text Revision. (APA, 2000). In certain embodiments, the invention provides a method for treating an anxiety disorder and reducing suicidality in a subject, by coordinately administering an anxiolytic drug effective to treat the anxiety disorder, and a lithium agent effective to reduce suicidality in the treated subject. Exemplary anxiety disorders in this context include panic disorder, agoraphobia, generalized anxiety disorder, specific phobia, social phobia, obsessive-compulsive disorder, acute stress disorder, post-traumatic stress disorder (PTSD), adjustment disorders with anxious features, anxiety disorders due to general medical conditions, substance-induced anxiety disorders, and a residual category of anxiety disorder not otherwise specified. Substance-induced anxiety disorders can be induced by alcohol, amphetamines, caffeine, cannabis, cocaine, hallucinogens, inhalants, phencyclidine, sedatives, hypnotics, anxiolytics and other substances.

Anxiety disorders are among the few mental disorders for which animal models are available. Researchers can reproduce symptoms of human anxiety in test animals by manipulating physical or psychosocial stressors. These animal models provide additional means for determining effective coordinate treatment methods and combinatorial formulations of the invention.

In more detailed embodiments of the invention, generalized anxiety is treated, which disorder is typically defined as an extended period (e.g. at least six months) of excessive anxiety or worry with symptoms on most days of this period. In other detailed embodiments, the methods and compositions of the invention are employed to treat a panic disorder, defined as the presence of recurrent panic attacks followed by at least one month of persistent concern about having another panic attack. A “panic attack” is a discrete period in which there is a sudden onset of intense apprehension, fearfulness or terror. During a panic attack, the individual may experience a variety of symptoms including palpitations, sweating, trembling, shortness of breath, chest pain, nausea and dizziness. Panic disorder may occur with or without agoraphobia. Alternate anxiety disorders amenable to treatment according to the invention include phobias; for example agoraphobia, specific phobias and social phobias.

Agoraphobia is characterized by anxiety about being in places or situations from which escape might be difficult or embarrassing, for example on an airplane, or in which help may not be available in the event of a panic attack. Agoraphobia may occur without history of a panic attack. A “specific phobia” is characterized by clinically significant anxiety provoked by exposure to a specific feared object or situation. Specific phobias include the following subtypes: animal type, cued by animals or insects; natural environment type, cued by objects in the natural environment, for example storms, heights or water; blood-injection-injury type, cued by the sight of blood or an injury or by seeing or receiving an injection or other invasive medical procedure; situational type, cued by a specific situation such as public transportation, tunnels, bridges, elevators, flying, driving or enclosed spaces; and other type where fear is cued by other stimuli. A “social phobia” is characterized by clinically significant anxiety provoked by exposure to certain types of social or performance circumstances. Social phobia may also be referred to as social anxiety disorder.

In addition to the above-described behavioral screening tests for measuring anxiolytic efficacy, the methods and compositions of the invention can be further demonstrated using animal models of anxiolytic activity. One of ordinary skill in the art will appreciate there are a number of animal models available for assessing anti-anxiety effects of compounds and methods of the invention. Two pharmacologically validated animal models of anxiety are the elevated zero maze test, and the isolation-induced ultrasonic emission test (Bickerdike, M. J. et al., Eur. J. Pharmacol., 271, 403 411 (1994); Shepherd, J. K. et al., Psychopharmacology, 116, 56 64 (1994)). Clinically used anxiolytic drugs, such as the benzodiazepines, increase the proportion of time spent in, and the number of entries made into, the open compartments. A second test for an anti-anxiety compound is the ultrasonic vocalization emission model, which measures the number of stress-induced vocalizations emitted by rat pups removed from their nest (Insel, T. R. et al., Pharmacol. Biochem. Behav., 24, 1263 1267 (1986); Miczek, K. A. et al., Psychopharmacology, 121, 38 56 (1995); Winslow, J. T. et al., Biol. Psychiatry, 15, 745 757 (1991).

Additional methods and compositions of the invention are directed toward treatment and prevention of seizure disorders and associated suicidality. In one exemplary embodiment, the invention provides methods and compositions for treating epilepsy and reducing suicide by coordinately administering an anticonvulsant CNS therapeutic and a lithium agent, wherein the CNS therapeutic agent and lithium agent are combinatorially effective to treat the epileptic condition and reduce suicidality in the treated subject.

Within other embodiments of the invention, the CNS disorder for coordinate treatment with lithium to treat the CNS condition and reduce suicidality according to the invention is an appetite or eating disorder. Appetite and eating disorders within this aspect of the invention can include, for example, bulimia nervosa, anorexia nervosa, obesity, and eating disorders not otherwise specified (NOS). In related embodiments of the invention, methods and compositions are provided for reducing appetite, body fat or body weight, for treating or preventing overeating, obesity or overweight, for reducing food intake, and/or for treating an appetite disorder in a mammal with a concomitant reduction of suicidality in treated subjects.

Compounds and methods of the invention can be administered to an animal to determine whether they affect food intake and body weight, body fat, appetite, food seeking behavior, or modulate fatty acid oxidation. Methods of conducting such tests are known to those of ordinary skill in the art (see, e.g., U.S. patent application Ser. No. 60/336,289. Animals can be, for example, obese or normal guinea pigs, rats, mice, or rabbits. Suitable rat model animals include, for example, Zucker rats. Suitable mice include mice with diet induced obesity. Other methods of assessing appetitive behavior are known to one of ordinary skill in the art. For instance, Maruani et al. (U.S. Pat. No. 6,344,474) teach two such assays. One method of assessing the effect on appetite behavior is to administer a FAAH inhibitor to a rat and assess its effect on the intake of a sucrose solution. This method is taught in W. C. Lynch et al., Physiol. Behav., 1993, 54, 877 880.

Within other embodiments of the invention, the CNS disorder for coordinate treatment using a CNS therapeutic drug in combination with lithium is an addictive disorder. Addictive disorders amenable to the treatment methods and compositions of the invention include, for example, alcohol-related addictive disorders, nicotine-related addictive disorders, amphetamine-related addictive disorders, cannabis-related addictive disorders, cocaine-related addictive disorders, hallucinogen use disorders, inhalant-related addictive disorders, opioid-related addictive disorders, and other addictive disorders known in the art.

Yet additional methods and compositions of the invention are effective for treating psychotic disorders, Alzheimer's disease, Parkinson's disease, epilepsy, seizure disorders, tic disorders, attention deficit/hyperactivity disorders, learning disorders, impulse control disorders, oppositional defiant disorders, conduct disorders, abnormal sexual behaviors, schizoid behaviors, somatization, sleep disorders, autism spectrum disorders, and other mood disorders and mental illnesses that differ from the depressive, anxiety, eating and addictive disorders set forth above. Exemplary psychotic disorders amenable to treatment with reduced suicidality employing the methods and compositions of the invention include, but are not limited to, schizophrenia, schizophreniform diseases, acute mania, schizoaffective disorders, depression with psychotic features, and other psychotic disorders known in the art and associated with increased risks of suicide.

Within the coordinate treatment methods and combinatorial formulations of the invention, a wide variety of CNS therapeutic agents may be selected, including drugs from each general class of anti-depressant, mood-stabilizing, anxiolytic, anticonvulsant, antipsychotic, anti-addictive, and appetite suppressant drugs. (See, e.g., R J. Baldessarini in Goodman & Gilman's The Pharmacological Basis of Therapeutics, 9th Edition, Chapter 18, McGraw-Hill, 1996 for a review).

In certain embodiments of the invention, the CNS therapeutic agent is an anti-depressant drug, which may include, for example, any species within the broad families of tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), and indeterminate (atypical) anti-depressants.

In more detailed embodiments of the invention, the methods and compositions employ an SSRI anti-depressant. Within exemplary embodiments, the SSRI is citalopram.

Other classes and species of drug agents have been developed and used, with variable success, to treat depression and other CNS disorders contemplated herein, and in general these other classes and species of drugs can be employed within the coordinate treatment methods and combinatorial formulations of the invention. Among the additional classes of drugs for treating CNS disorders as contemplated herein within the coordinate treatment methods and combinatorial compositions of the invention include, mood-stabilizing, anxiolytic, anticonvulsant, antipsychotic, anti-addictive, and appetite suppressant drugs. The efficacy and safety profiles of these different classes of drugs are generally well-known in the art.

Depression has been treated in recent years usually by the combination of anti-depressants selected from one of three major groups of compounds. These are the tricyclic and tetracyclic anti-depressants and related compounds (“tricyclics”); the monoamine oxidase inhibitors (MAOIs); and the selective or partially selective serotonin uptake inhibitors (SSRIs). The tricyclics, including imipramine, desipramine, amitriptyline, nortriptyline, and doxepin, have many and complex mechanisms of action, and are associated with many side effects, including cardiovascular side effects and toxicity such that they can be used for suicide attempts. The MAOIs inhibit one of the main enzymes involved in the degradation of catecholamines and also can have many side effects. Both the tricyclics and the MAOIs must be used with great caution in patients with cardiovascular disease. The SSRIs have a relatively more selective action in inhibiting the reuptake of serotonin by nerve terminals and usually have fewer side effects than the other groups. Recently, a further group of compounds known as noradrenaline reuptake inhibitors (NRIs), which constitute a new class of anti-depressants, have been approved for clinical use. Additionally, multiple monoamine reuptake inhibitors, e.g., that inhibit both serotonin and norepinephrine reuptake (SNRIs), are useful within this aspect of the invention.

The most established drug treatment for the management of depressive illness is the tricyclic anti-depressants. For instance, depressed patients with prominent sleep disturbance and anxiety may be treated with a sedating tricyclic anti-depressant such as amitriptyline; for other patients, less sedating compounds such as imipramine or desipramine can be used. As well as inhibiting the uptake of noradrenaline and 5-hydroxytriptamine, tricyclic anti-depressants also possess antagonist properties at a variety of neurotransmitter receptors, including muscarinic cholnergic receptors, α₁-adrenoceptors and H₁-histamine receptors. These receptor antagonist effects account for much of the side-effect profile of the tricyclic anti-depressants, and in particular, their anticholinergic side-effects which are particularly troublesome in patients with prostatic enlargement or glaucoma. Other side-effects may include dry mouth, tachycardia, difficulty in visual accommodation, constipation, urinary retention, sexual dysfunction, cognitive impairment, postural hypotension, and/or weight gain.

Monoamine oxidase inhibitors are generally prescribed for patients who have failed to respond to tricyclic anti-depressants or electroconvulsive therapy. As with tricyclic anti-depressants, there are a number of side-effects associated with the use of MAOIs, including dizziness, muscular twitching, insomnia, confusion, mania, tachycardia, postural hypotension, hypertension, dry mouth, blurred vision, impotence, peripheral oedema, hepatocellular damage and leucopenia.

Of the new classes of anti-depressant, selective serotonin reuptake inhibitors (SSRIs) are increasingly prescribed, particularly in patients where the use of tricyclic anti-depressants is contraindicated because of their anticholinergic and cardiotoxic effects. SSRIs such as fluoxetine, fluvoxamine, sertraline and paroxetine are generally non-sedating. Furthermore, SSRIs do not typically stimulate appetite and may therefore be appropriate in patients in whom weight gain would be undesirable. However, SSRIs are not without their own side-effects, including nausea, diarrhea, dry mouth, reduced appetite, dyspepsia, vomiting, headache, nervousness, insomnia, anxiety, tremor, dizziness, fatigue, decreased libido, pharyngitis, dyspnoea, skin rash and sexual dysfunction.

Additional drugs for treatment of depression and other mood disorders include monoamine reuptake inhibitors, which generally function by increasing extracellular levels of monoamine neurotransmitters, for example, norepinephrine, serotonin and/or dopamine. The increase in neurotransmitter levels trigger a cascade of intracellular neurochemical changes that can eventually lead to desired therapeutic CNS effects. (See, e.g., Bymaster et al., Neuropsychopharmacology 27:699-711. (2002); Richelson, J. Clin. Psychiatry. 64:5-12. (2003)). These monoamine reuptake inhibitors have potential uses as medications in a wide variety of CNS disorders ranging from depression, anxiety, eating disorders, and drug or alcohol addictive disorders. Exemplary monoamine reuptake inhibitors in this context include SSRIs (e.g. fluoxetine (Prozac®) and sertraline (Zoloft®), and multiple monoamine reuptake inhibitors, e.g., that inhibit both serotonin and norepinephrine reuptake (SNRIs; e.g. venlafaxine (Effexor®), and duloxetine (Cymbalta®), which have been widely applied to treat depression and anxiety disorders).

Within exemplary embodiments of the invention one or more of the anti-depressant drugs identified in Table 1 below is coordinately administered or combinatorially formulated with lithium to treat a CNS disorder (e.g., depression or anxiety) and decrease suicidality among treated patients. Single drugs, or multiple drugs from one or more of the indicated drug classes, may be coadministered, simultaneously or sequentially, with the lithium agent, which may be combinatorially formulated with the CNS therapeutic drug or provided in a separate dosage form.

TABLE 1
EXEMPLARY ANTI-DEPRESSANT DRUGS FOR COORDINATE
ADMINISTRATION WITH LITHIUM

SSRIs

Celexa ® (citalopram)
Lexapro ® (excitalopram oxalate)
Luvox ® (fluvoxamine)
Paxil ® (paroxetine)
Prozac ® (fluoxetine)
Symbyax ®
Zoloft ® (sertraline)

SNRIs

Cymbalta ® (duloxetine)
Effexor ® (venlafaxine)
Pristiq ® (desvenlafaxine)
Tricyclics
Adapin ® (doxepin)
Anafranil ® (clomipramine)
Elavil ® (amitriptyline)
Endep ® (amitriptyline)
Ludiomil ® (maprotiline)
Norpramin ® (desipramine)
Pamelor ® (nortryptyline)
Pertofrane ® (desipramine)
Sinequan ® (doxepin)
Surmontil ® (trimipramine)
Tofranil ® (imipramine)
Vivactil ® (protriptyline)

Other Approved Anti-depressants

Remeron ® (mirtazapine)
Vilazodone ®
Wellbutrin ® (bupropion)

Pending FDA Approval

Agomelatine ®

In other detailed embodiments of combinatorial formulations and coordinate treatment methods of the invention, examples of useful anti-depressant agents include, but are not limited to, one or more of the following: MAOIs, such as phenelzine, nortriptyline, selegiline and tranylcypromine; SSRIs, such as paroxetine, fluoxetine, citalopram, trazodone, fluvozamine and sertraline; Tricyclic anti-depressants, such as amitriptyline, desipramine, clomipramine, doxepine, trimipramine, amoxapine, protripyline and imipramine; Tetracyclic anti-depressants; Norepinephrine uptake inhibitors; Selective noradrenaline reuptake inhibitors; Serotonin and norepinephrine reuptake inhibitors, such as venlafaxine and duloxetine; and other anti-depressant agents such as maprotiline, nefazodone, and bupropion. In additional detailed embodiments the combinatorial formulations and coordinate treatment methods of the invention employ one or more useful CNS therapeutic agents selected from the following: SSRI's, such as Lexapro® (escitalopram HBr; indicated to treat depression and generalized anxiety disorder Celexa® (citalopram), Prozac®, Luvox® (fluvoxamine; also indicated to treat obsessive symptoms), Zoloft® (sertraline; also indicated to treat post-traumatic stress syndrome); Tricyclics, such as Amitriptyline, Desipramine, Nortriptyline; SSNRIs, such as Cymbalta® (Duloxetine), Effexor®, and desvenlafaxine; Tetracyclics, such as Remeron® (mirtazepine); MAOIs, such as Nardil® (phenelzine), and Parnate® (tranylcypromine); Serzone® (nefazodone; a phenylpiperazine); Trazodone® (a triazolopyridine); and Wellbutrin® (bupropion; an aminoketone). In additional detailed embodiments the combinatorial formulations and coordinate treatment methods of the invention employ one or more useful CNS therapeutic agents selected from the following: amitriptyline; amoxapine; aripiprazole; atomoxetine; bupropion; citalopram; clomipramine; desipramine; desvenlafaxine; dothiepin; doxepin; duloxetine; escitalopram; fluoxetine; fluvoxamine; imipramine; isocarboxazid; lofepramine; maprotiline; milnacipran; mirtazapine; moclobemide; nefazodone; notriptyline; paroxetine; phenelzine; protriptyline; quetiapine; reboxetine; selegiline; sertraline; tianeptine; tranylcypromide; trazodone; trimipramine; and venlafaxine.

In other detailed combinatorial formulations and coordinate treatment methods of the present invention, the CNS therapeutic agent is an anxiolytic drug agent. As in the case of many mood disorders, including depressive disorders, certain anxiety disorders have been linked to abnormal neurophysiology such as defects in signal transduction involving serotonin and dopamine (Nakamura et al., 2001). Thus, examples of useful anxiolytic agents for use within the invention include, but are not limited to: monamine reuptake inhibitors, including SSRI's, multiple monamine reuptake inhibitors, including SSNRI's and other compounds that regulate neurotransmitter and/or neurotransmitter receptor physiology. Among the other useful anxiolytic agents for use within the invention are benzodiazepines, such as alaprazolam, chlordiazepoxide, clonazepam, chlorazepate, diazepam, lorazepam, oxazepam and prazepam; non-benzodiazepine agents, such as buspirone; and tranquilizers, such as barbituates. Long-term administration of benzodiazepine drugs can induce lowering of cognition (Rickels et al., 1983) and physical dependence and tolerance (Ashton, 1984). Apart from the risks of drug dependency, benzodiazepines are also associated with a number of side-effects including increased hostility and irritability, vivid or disturbing dreams, weight gain, skin rash, nausea, headache, impairment of sexual function, vertigo, and lightheadedness.

Benzodiazepines, anti-depressants, selective serotonin reuptake inhibitors and the azapirone agonist of the serotonin 1A receptor, buspirone (Lydiard et al., 1996) have been used with some success in the treatment of anxiety and anxiety disorders and are therefore contemplated for effective use within the methods and compositions of the invention.

Additional CNS therapeutic agents for use within the invention are any of a variety of known drugs effective for regulating appetite and/or controlling body weight. Obesity is often caused by defects in normal appetite control and/or metabolic control in the body. A common target for treating obesity is to induce suppression of appetite by increasing the level of brain serotonin known to be an appetite suppressing neurotransmitter. If the secretion of serotonin is increased in the brain, appetite may be suppressed. This relationship was actually found in the process of treating depression patients, where many depression patients were administered anti-depressant drugs that also increase serotonin, yielding decreases appetite in treated subjects. Thus, compounds that selectively inhibit reuptake of serotonin and/or norepinephrine in pre-synaptic neurons can increase the level of serotonin in the synapse leading to effective appetite reduction in treated subjects (Richard J et al., J Nutr Biochem 9, 511-515, 1998; Ljung, T et al., J of internal medicine 250(30), 219-224, 2001). Exemplary CNS therapeutic drugs in this context include fenfluramine, dexfenfluramine and sibutramine which are prescribed to obesity patients and have an activity to selectively inhibit reuptake of serotonin and norepinephrine in pre-synaptic neuron (thereby increasing levels of serotonin in the synapse) (Richard J et al., J Nutr Biochem 9, 511-515, 1998; Ljung, T et al., J of internal medicine 250(30), 219-224, 2001). Other currently prescribed drugs for appetite control and/or obesity include orlistat, which reportedly reduces the amount of dietary fat absorbed from the intestine; sibutramine, which reportedly suppresses appetite by inhibiting re-uptake of norepinephrine and serotonin; and phentermine, which reportedly suppresses appetite by stimulating release of norepinephrine.

Other CNS therapeutic drugs for use within the invention include any one of more of a variety of drugs indicated for treatment of addictive disorders, including various species of drugs described above for treatment of depression and/or anxiety disorders.

In certain embodiments of combinatorial formulations and coordinate treatment methods provided herein, the CNS therapeutic agent is an anti-attention-deficit-disorder treatment agent. Examples of useful anti-attention-deficit-disorder agents for use in these embodiments include, but are not limited to, ethylphenidate; dextroamphetamine; tricyclic anti-depressants, such as imipramine, desipramine, and nortriptyline; and psychostimulants, such as pemoline and deanol.

In additional embodiments of combinatorial formulations and coordinate treatment methods provided herein, the CNS therapeutic agent is an anti-addictive-disorder agent. Examples of useful anti-addictive-disorder agents include, but are not limited to, tricyclic anti-depressants; glutamate antagonists, such as ketamine HCl, dextromethorphan, dextrorphan tartrate and dizocilpine; degrading enzymes, such as anesthetics and aspartate antagonists; GABA agonists, such as baclofen and muscimol HBr; reuptake blockers; degrading enzyme blockers; glutamate agonists, such as D-cycloserine, carboxyphenylglycine, L-glutamic acid, and cis-piperidine-2,3-dicarboxylic acid; aspartate agonists; GABA antagonists such as gabazine (SR-95531), saclofen, bicuculline, picrotoxin, and; and dopamine antagonists, such as spiperone HCl, haloperidol, (+) apomorphine HCl and (−) sulpiride.

In other embodiments of combinatorial formulations and coordinate treatment methods provided herein, the CNS therapeutic agent is an alcohol addiction treatment agent. Examples of useful alcohol addiction treatment agents include, but are not limited to, disulfiram and naltrexone.

In other embodiments of combinatorial formulations and coordinate treatment methods provided herein, the CNS therapeutic agent is an anti-nicotine agent. Examples of useful anti-nicotine agents include, but are not limited to, clonidine, mecamylamine and varenicline.

In other embodiments of combinatorial formulations and coordinate treatment methods provided herein, the CNS therapeutic agent is used in the treatment of opiate addictions. Examples of useful additional opiate addiction treatment agents include, but are not limited to, methadone, clonidine, lofexidine, levomethadyl acetate HCl, naltrexone, and buprenorphine.

In other embodiments of combinatorial formulations and coordinate treatment methods provided herein, the CNS therapeutic agent is cocaine addiction treatment agent Examples of useful cocaine addiction treatment agents include, but are not limited to, desipramine, amantadine, fluoxidine, and buprenorphine.

In other embodiments of combinatorial formulations and coordinate treatment methods provided herein, the CNS therapeutic agent is a phencyclidine induced psychosis treatment agent. Examples of useful phencyclidine induced psychosis treatment agents include, but are not limited to, haloperidol.

In other embodiments of combinatorial formulations and coordinate treatment methods provided herein, the CNS therapeutic agent is an appetite suppressant. Examples of useful appetite suppressants include, but are not limited to, fenfluramine, phenylpropanolamine, and mazindol.

Additional CNS therapeutic drugs for use within the invention include any one of more of a variety of drugs indicated for treatment of psychotic disorders, Alzheimer's disease, Parkinson's disease, epilepsy, seizure disorders, tic disorders, attention deficit/hyperactivity disorders, learning disorders, impulse control disorders, oppositional defiant disorders, conduct disorders, abnormal sexual behaviors, schizoid behaviors, somatization, sleep disorders, autism spectrum disorders, and other mood disorders and mental illnesses that differ from the depressive, anxiety, eating and addictive disorders set forth above. Exemplary CNS therapeutic drugs in these contexts include various species of drugs described above for treatment of depression and/or anxiety disorders, as well as other drugs known in the art having anti-depressant, mood-stabilizing, anxiolytic, anticonvulsant, antipsychotic, anti-addictive, and/or appetite suppressant activities. (See, e.g., R J. Baldessarini in Goodman & Gilman's The Pharmacological Basis of Therapeutics, 9th Edition, Chapter 18, McGraw-Hill, 1996 for a review)

In related embodiments of combinatorial formulations and coordinate treatment methods provided herein, the CNS therapeutic agent is Parkinson's disease treatment agent. Examples of useful Parkinson's-disease treatment agents include, but are not limited to dopamine precursors, such as levodopa, and L-tyrosine; neuroprotective agents; dopamine agonists; dopamine reuptake inhibitors; anticholinergics such as amantadine and memantine; and 1,3,5-trisubstituted adamantanes, such as 1-amino-3,5-dimethyl-adamantane (See, U.S. Pat. No. 4,122,193).

Within other aspects of the invention, the lithium is administered in the form of a lithium salt, such as lithium citrate or lithium carbonate. Other lithium salts for use within the invention may include, for example any feasible salts of lithium selected from, for example, sulfate salt (Li₂SO₄), the oxybutyrate salt (C₄H₉LiO₃) aspartate and the orotate salt, bromide salt (LiBR), chloride salt (LiCl), fluoride salts (LiF), iodide salts (LII), phosphate salts (Li₃PO₄), sulfate salts (LI₂SO₄), acetate salts (LiC₂H₃O₂), hydrogen sulphate, nitrate, hydrogen phosphate, lactate, succinate, tartrate, maleate, fumarate, mandelate, acetate, dichloroacetate, trifluoroacetate, oxalate, formate, sulfonate, arginate, asparginate, glutamate, tartrate, and gluconate salts. In alternate embodiments, the lithium is provided in a complex or admixture with a binding partner or carrier, for example a sustained release binding partner or carrier. Examples of such carriers include, without limitation, hydrophobic counter ions such as decanoate, palmitate or oleate, differentially encapsulated beads, liposomal formulations that may include surface lectins for delayed passage through the intestinal tract or poly anionic resins (e.g. poly styrene sulphonates). Delayed release preparations may also include intramuscular or subcutaneous biodegradable polymers (e.g. poly-(L)-lactate) or poly anionic resins (e.g. poly styrene sulphonates).

In more detailed embodiments of the invention, the lithium is administered in an antisuicidal effective dosage of from about 50 mg to about 2000 mg. In other embodiments, the lithium is administered in a surprisingly low, antisuicidal effective dosage of from about 100 mg to about 500 mg, in other embodiments in an antisuicidal effective dosage of about 500 mg or less, and in still other embodiments of antisuicidal effective dosage of about 300 mg or less. Notably, recommended dosages of lithium for its only approved indication (bipolar disorder) are in a much higher range of from about 900 mg to 1800 mg per day.

Within various aspects of the invention, the CNS therapeutic agent and lithium may each be administered by any of a variety of delivery routes and modes, which may be the same or different for each agent. In alternate embodiments, the CNS therapeutic drug and/or lithium is administered by a mode of delivery selected from oral, buccal, nasal, aerosol, topical, transdermal, mucosal, or injectable. In exemplary embodiments, both the CNS therapeutic agent and lithium are administered orally. In other embodiments, one or both of the CNS drug and lithium is/are delivered in a sustained or extended release formulation, for example in a sustained release, oral or intravenous formulation.

The amount, timing and mode of delivery of compositions of the invention comprising an effective amount of a CNS therapeutic compound and an antisuicidal effective amount of a lithium agent will be routinely adjusted on an individual basis, depending on such factors as weight, age, gender, and condition of the individual, the acuteness of the targeted CNS disorder and/or related symptoms, whether the administration is prophylactic or therapeutic, and on the basis of other factors known to effect drug delivery, absorption, pharmacokinetics, including half-life, and efficacy. An effective dose or multi-dose treatment regimen for the CNS therapeutic compounds of the invention will ordinarily be selected to approximate a minimal dosing regimen that is necessary and sufficient to substantially prevent or alleviate one or more symptom(s) of the targeted CNS disorder as described herein. Exemplary suggested dosage ranges for selected drugs for use within certain embodiments of the invention are provided below in Table 2, for illustrative purposes. Additional exemplary dosage ranges are provided below in Table 3 for selected drugs formulated for sustained delivery within additional embodiments of the invention, also for illustrative purposes.

TABLE 2
Exemplary Effective Dosage Ranges For Selected
Oral Anti-Depressant Medications

SSRIs

| | | |
|---|---|---|
| Celexa ® (citalopram) | 20-40 | mg |
| Lexapro ® (excitalopram oxalate) | 10-20 | mg |
| Luvox ® (fluvoxamine) | 100 | mg |
| Paxil ® (paroxetine) | 20-40 | mg |
| Prozac ® (fluoxetine) | 20 | mg |
| Symbyax ® (Zyprexa & Prozac) | 12.5-50 | mg |
| Zoloft ® (sertraline) | 50-200 | mg |

SNRIs

| | | |
|---|---|---|
| Cymbalta ® (duloxetine) | 60-120 | mg |
| Effexor ® (venlafaxine) | 75-375 | mg |
| Pristiq ® (desvenlafaxine) | 50 | mg |

Tricyclics

| | | |
|---|---|---|
| Adapin ® (doxepin) | 150 | mg |
| Anafranil ® (clomipramine) | 150 | mg |
| Elavil ® (amitriptyline) | 150 | mg |
| Endep ® (amitriptyline) | 150 | mg |
| Ludiomil ® (maprotiline) | 100 | mg |
| Norpramin ® (desipramine) | 150 | mg |
| Pamelor ® (nortryptyline) | 75 | mg |
| Pertofrane ® (desipramine) | 150 | mg |
| Sinequan ® (doxepin) | 150 | mg |
| Surmontil ® (trimipramine) | 150 | mg |
| Tofranil ® (imipramine) | 150 | mg |
| Vivactil ® (protriptyline) | 75 | mg |

Other Approved Anti-depressants

| | | |
|---|---|---|
| Remeron ® (mirtazapine) | 20-40 | mg |
| Vilazodone ® | 40 | mg |
| Wellbutrin ® (bupropion) | 300 | mg |

Pending FDA Approval

| | | |
|---|---|---|
| Agomelatine ® | 50-100 | mg |

TABLE 3
Exemplary Effective Dosage Ranges for Selected CNS Therapeutic
Compounds in Sustained Release Formulations/Methods

SSRIs

| | Celexa ® (citalopram) | 60-1000 | mg |
|---|---|---|---|
| | Lexapro ® (excitalopram oxalate) | 60-1000 | mg |
| vLuvox ® (fluvoxamine) | | 100-2000 | mg |
| | Paxil ® (paroxetine) | 60-100 | mg |
| | Prozac ® (fluoxetine) | 60-50 | mg |
| | Symbyax ® (Zyperxa & Prozac) | 60-500 | mg zyprexa |
| | | 60-1000 | mg prozac |
| | Zoloft ® (sertraline) | 200-2500 | mg |

SNRIs

| | | |
|---|---|---|
| Cymbalta ® (duloxetine) | 100-2000 | mg |
| Effexor ® (venlafaxine) | 300-3000 | mg |
| Pristiq ® (desvenlafaxine) | 100-2000 | mg |

Tricyclics

| | | |
|---|---|---|
| Adapin ® (doxepin) | 200-1000 | mg |
| Anafranil ® (clomipramine) | 100-1000 | mg |
| Elavil ® (amitriptyline) | 150-2000 | mg |
| Endep ® (amitriptyline) | 150-2000 | mg |
| Ludiomil ® (maprotiline) | 100-1000 | mg |
| Norpramin ® (desipramine) | 100-1000 | mg |
| Pamelor ® (nortryptyline) | 100-1000 | mg |
| Pertofrane ® (desipramine) | 100-1000 | mg |
| Sinequan ® (doxepin) | 100-1000 | mg |
| Surmontil ® (trimipramine) | 100-1000 | mg |
| Tofranil ® (imipramine) | 100-100 | mg |
| Vivactil ® (protriptyline) | 50-500 | mg |

Other Approved Anti-depressants

| | | |
|---|---|---|
| Remeron ® (mirtazapine) | 50-500 | mg |
| Vilazodone ® | 50-1000 | mg |
| Wellbutrin ® (bupropion) | 300-3000 | mg |

Pending FDA Approval

| | | |
|---|---|---|
| Agomelatine ® | 100-2000 | mg |

These and other effective unit dosage amounts of either or both of the CNS therapeutic agent and/or lithium may be administered in a single dose, or in the form of multiple daily, weekly or monthly doses, for example in a dosing regimen comprising from 1 to 5, or 2-3, doses administered per day, per week, or per month. In exemplary embodiments, exemplary dosages of selected drugs as illustrated above are administered one, two, three, or four times per day. In more detailed embodiments, specific dosages within the specified exemplary ranges above are administered once, twice, or three times daily. In alternate embodiments, dosages are calculated based on body weight, and may be administered, for example, in amounts as exemplified above adjusted for body weight.

Pharmaceutical dosage forms of a compound of the present invention may optionally include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

The compositions of the invention for treating CNS disorders, including depression, and reducing suicidality can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents will often be biologically inactive and can be administered to patients without causing deleterious side effects or interactions with the active agent.

A CNS therapeutic compound and/or lithium agent of the present invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets and capsules, which may be prepared by any conventional method of preparing pharmaceutical oral unit dosage forms can be utilized in preparing oral unit dosage forms. Oral unit dosage forms, such as tablets or capsules, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate. Such rapidly acting dosage forms would be useful, for example, in the prevention or treatment of acute attacks of panic disorder.

The CNS therapeutic compound and/or lithium agent of the invention can be prepared and administered in any of a variety of inhalation or nasal delivery forms known in the art. Devices capable of depositing aerosolized formulations of a CNS therapeutic compound and/or lithium agent of the invention in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of seizures or panic disorder. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of a compound of the present invention, and any additional active or inactive ingredient(s).

Intranasal delivery permits the passage of active compounds of the invention into the blood stream directly after administering an effective amount of the compound to the nose, without requiring the product to be deposited in the lung. In addition, intranasal delivery can achieve direct, or enhanced, delivery of the active CNS therapeutic compound and/or lithium agent to the CNS. In these and other embodiments, intranasal administration of the compounds of the invention may be advantageous for treating a variety of CNS disorders., including depression, by providing for rapid absorption and CNS delivery.

For intranasal and pulmonary administration, a liquid aerosol formulation will often contain an active compound of the invention combined with a dispersing agent and/or a physiologically acceptable diluent. Alternative, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle suitable of a sufficiently small particle diameter, e.g., in a range of from about 2-5 microns, for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional compositions and methods of the invention are provided for topical administration of a CNS therapeutic compound and/or lithium agent of the present invention for treating CNS disorders, including depression, and reducing suicidality. Topical compositions may comprise a compound of the present invention and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a compound of the present invention dissolved or dispersed in water or other solvent or liquid to be incorporated in the topical composition or delivery device. It can be readily appreciated that the transdermal route of administration may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example 24 hours. A once-daily transdermal patch will be particularly useful for patients suffering from selected CNS disorders, such as generalized anxiety disorder.

Yet additional formulations of a compound of the present invention are provided for parenteral administration, including aqueous and non-aqueous sterile injection solutions which may optionally contain anti-oxidants, buffers, bacteriostats and/or solutes which render the formulation isotonic with the blood of the mammalian subject; aqueous and non-aqueous sterile suspensions which may include suspending agents and/or thickening agents; dispersions; and emulsions. The formulations may be presented in unit-dose or multi-dose containers. Pharmaceutically acceptable formulations and ingredients will typically be sterile or readily sterilizable, biologically inert, and easily administered. Parenteral preparations typically contain buffering agents and preservatives, and may be lyophilized for reconstitution at the time of administration.

Parenteral formulations may also include polymers for extended release following parenteral administration. Such polymeric materials are well known to those of ordinary skill in the pharmaceutical compounding arts. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

Within exemplary compositions and dosage forms of the invention, the CNS therapeutic compound and/or lithium agent for treating CNS disorders and reducing suicidality is/are administered in an extended release or sustained release formulation. In these formulations, the sustained release composition of the formulation provides therapeutically effective plasma levels of the CNS therapeutic compound and/or lithium agent over a sustained delivery period of approximately 8 hours or longer, or over a sustained delivery period of approximately 18 hours or longer, up to a sustained delivery period of approximately 24 hours or longer.

In exemplary embodiments, the CNS therapeutic compound and/or lithium agent is/are combined with a sustained release vehicle, matrix, binder, or coating material. As used herein, the term “sustained release vehicle, matrix, binder, or coating material” refers to any vehicle, matrix, binder, or coating material that effectively, significantly delays dissolution of the active compound in vitro, and/or delays, modifies, or extends delivery of the active compound into the blood stream (or other in vivo target site of activity) of a subject following administration (e.g., oral administration), in comparison to dissolution and/or delivery provided by an “immediate release” formulation, as described herein, of the same dosage amount of the active compound. Accordingly, the term “sustained release vehicle, matrix, binder, or coating material” as used herein is intended to include all such vehicles, matrices, binders and coating materials known in the art as “sustained release”, “delayed release”, “slow release”, “extended release”, “controlled release”, “modified release”, and “pulsatile release” vehicles, matrices, binders and coatings.

In one aspect, the current invention comprises an oral sustained release dosage composition for administering a CNS therapeutic compound and/or lithium agent according to the invention. In a related aspect, the invention comprises a method of reducing one or more side effects that attend administration of an oral dosage form of a CNS therapeutic compound and/or lithium agent compound by employing a sustained release formulation. Within these methods, a CNS therapeutic compound and/or lithium agent is provided in a sustained release oral dosage form and the dosage form is introduced into a gastrointestinal tract of a mammalian subject presenting with a CNS disorder amenable to treatment using the subject CNS therapeutic drug, by having the subject swallow the dosage form. The method further includes releasing the active CNS therapeutic compound and/or lithium agent in a sustained, delayed, gradual or modified release delivery mode into the gastrointestinal tract (e.g., the intestinal lumen) of the subject over a period of hours, during which the active CNS therapeutic compound and/or lithium agent reach(es), and is/are sustained at, therapeutic concentration(s) in a blood plasma, tissue, organ or other target site of activity (e.g., a central nervous system (CNS) tissue, fluid or compartment) in the patient. When following this method, the side effect profile of the CNS therapeutic compound and/or lithium agent is less than a side effect profile of an equivalent close of the CNS therapeutic compound and/or lithium agent administered in an immediate release oral dosage form.

In certain embodiments, the CNS therapeutic compound and/or lithium agent is/are released from the sustained release compositions and dosage forms of the invention and delivered into the blood plasma or other target site of activity in the subject at a sustained therapeutic level over a period of at least about 6 hours, often over a period of at least about 8 hours, at least about 12 hours, or at least about 18 hours, and in other embodiments over a period of about 24 hours or greater. By sustained therapeutic level is meant a plasma concentration level of at least a lower end of a therapeutic dosage range as exemplified herein. In more detailed embodiments of the invention, the sustained release compositions and dosage forms will yield a therapeutic level of a CNS therapeutic compound and/or lithium agent following administration to a mammalian subject in a desired dosage amount (e.g., 200, 400, 600, or 800 mg) that yields a minimum plasma concentration of at least a lower end of a therapeutic dosage range as exemplified herein over a period of at least about 6 hours, at least about 8 hours, at least about 12 hours, at least about 18 hours, or up to 24 hours or longer. In alternate embodiments of the invention, the sustained release compositions and dosage forms will yield a therapeutic level of CNS therapeutic compound and/or lithium agent following administration to a mammalian subject in a desired dosage amount (e.g., 200, 400, 600, or 800 mg) that yields a minimum plasma concentration that is known to be associated with clinical efficacy, over a period of at least about 6 hours, at least about 8 hours, at least about 12 hours, at least about 18 hours, or up to 24 hours or longer.

In certain embodiments, the active CNS therapeutic compound and/or lithium agent is/are released from the compositions and dosage forms of the invention and delivered into the blood plasma or other target site of activity in the subject in a sustained release profile characterized in that from about 0% to 20% of the active compound is released and delivered (as determined, e.g., by measuring blood plasma levels) within in 0 to 2 hours, from 20% to 50% of the active compound is released and delivered within about 2 to 12 hours, from 50% to 85% of the active compound is released and delivered within about 3 to 20 hours, and greater than 75% of the active compound is released and delivered within about 5 to 18 hours.

In more detailed embodiments of the invention, compositions and oral dosage forms of a CNS therapeutic compound and/or lithium agent are provided, wherein the compositions and dosage forms, after ingestion, provide a curve of concentration of the CNS therapeutic compound and/or lithium agent over time, the curve having an area under the curve (AUC) which is approximately proportional to the dose of the CNS therapeutic compound and/or lithium agent administered, and a maximum concentration (Cmax) that is proportional to the dose of the CNS therapeutic compound and/or lithium agent administered.

In other detailed embodiments, the Cmax of the active CNS therapeutic compound and/or lithium agent provided after oral delivery of a composition or dosage form of the invention is less than about 80%, often less than about 75%, in some embodiments less than about 60%, or 50%, of a Cmax obtained after administering an equivalent dose of the active compound in an immediate release oral dosage form.

Within exemplary embodiments of the invention, the compositions and dosage forms containing the CNS therapeutic compound and/or lithium agent and a sustained release vehicle, matrix, binder, or coating will yield sustained delivery of the active compound such that, following administration of the composition or dosage form to a mammalian treatment subject, the Cmax of the CNS therapeutic compound and/or lithium agent in the treatment subject is less than about 80% of a Cmax provided in a control subject after administration of the same amount of the CNS therapeutic compound and/or lithium agent in an immediate release formulation.

As used herein, the term “immediate release dosage form” refers to a dosage form of a CNS therapeutic compound and/or lithium agent wherein the active compound readily dissolves upon contact with a liquid physiological medium, for example phosphate buffered saline (PBS) or natural or artificial gastric fluid. In certain embodiments, an IR formulation will be characterized in that at least 70% of the active compound will be dissolved within a half hour after the dosage form is contacted with a liquid physiological medium. For example, at least 70% of the active compound in an IR lithium dosage form will dissolve within a half hour following contact of the dosage form with a liquid physiological medium in an art-accepted in vitro dissolution assay (e.g., using a USP 1 Apparatus, 20 mesh baskets, 75 rpm, and a dissolution medium comprised of 900 ml 0.01 N HCl at 37° C.±0.5° C.; or following an alternate USP basket method at 100 rpm in 700 ml Simulated Gastric Fluid (SGF) at 37° C. for 1 hour and thereafter switching to 900 ml with phosphate buffer to a pH of 7.5 at 37° C.). In alternate embodiments, at least 80%, 85%, 90% or more, or up to 100%, of the active compound in an IR dosage form will dissolve within a half hour following contact of the dosage form with a liquid physiological medium in an art-accepted in vitro dissolution assay. These general characteristics of an IR dosage form will often relate to powdered or granulated compositions of a CNS therapeutic compound and/or lithium agent in a capsulated dosage form, for example in a gelatin-encapsulated dosage form, where dissolution will often be relatively immediate after dissolution/failure of the gelatin capsule. In alternate embodiments, the IR dosage form may be provided in the form of a compressed tablet, granular preparation, powder, or even liquid dosage form, in which cases the dissolution profile will often be even more immediate (e.g., wherein at least 85%-95% of the active compound is dissolved within a half hour).

In additional embodiments of the invention, an IR dosage form will include compositions wherein the CNS therapeutic compound and/or lithium agent is not admixed, bound, coated or otherwise associated with a formulation component that substantially impedes in vitro or in vivo dissolution and/or in vivo bioavailability of the active compound. Within certain embodiments, the CNS therapeutic compound and/or lithium agent will be provided in an immediate release dosage form that does not contain significant amounts of a sustained release vehicle, matrix, binder or coating material. In this context, the term “significant amounts of a sustained release vehicle, matrix, binder or coating material” is not intended to exclude any amount of such materials, but an amount sufficient to impede in vitro or in vivo dissolution of a CNS therapeutic compound and/or lithium agent in a formulation containing such materials by at least 5%, often at least 10%, and up to at least 15%-20% compared to dissolution of the CNS therapeutic compound and/or lithium agent when provided in a composition that is essentially free of such materials.

In alternate embodiments of the invention, an IR dosage form of a CNS therapeutic compound and/or lithium agent may be any dosage form comprising the active compound which fits the FDA Biopharmaceutics Classification System (BCS) Guidance definition (see, e.g., http://www.fda.gov/cder/OPS/BCS_guidance.htm) of a “high solubility substance in a rapidly dissolving formulation.” In exemplary embodiments, an IR formulation of a CNS therapeutic compound and/or lithium agent formulation according to this aspect of the invention will exhibit rapid dissolution characteristics according to BCS Guidance parameters, such that at least approximately 85% of the CNS therapeutic compound and/or lithium agent in the formulation will go into a test solution within about 30 minutes at pH 1, pH 4.5, and pH 6.8.

The compositions, dosage forms and methods of the invention thus include novel tools for coordinate treatment of CNS disorders and suicidality providing for sustained release and/or sustained delivery of the CNS therapeutic compound and/or lithium agent. As used herein, “sustained release” and “sustained delivery” are evinced by a sustained, delayed, extended, or modified, in vitro or in vivo dissolution rate, in vivo release and/or delivery rate, and/or in vivo pharmacokinetic value(s) or profile.

The sustained release dosage forms of the present invention can take any form as long as one or more of the dissolution, release, delivery and/or pharmacokinetic property(ies) identified above are satisfied. Within illustrative embodiments, the composition or dosage form can comprise a CNS therapeutic compound and/or lithium agent combined with any one or combination of: a drug-releasing polymer, matrix, bead, microcapsule, or other solid drug-releasing vehicle; drug-releasing tiny timed-release pills or mini-tablets; compressed solid drug delivery vehicle; controlled release binder; multi-layer tablet or other multi-layer or multi-component dosage form; drug-releasing lipid; drug-releasing wax; and a variety of other sustained drug release materials as contemplated herein, or formulated in an osmotic dosage form.

The present invention thus provides a broad range of sustained release compositions and dosage forms comprising a CNS therapeutic compound and/or lithium agent, which in certain embodiments are adapted for providing sustained release of the active compound(s) following, e.g., oral administration. Sustained release vehicles, matrices, binders and coatings for use in accordance with the invention include any biocompatible sustained release material which is inert to the active agent and which is capable of being physically combined, admixed, or incorporated with the active compound. Useful sustained release materials may be dissolved, degraded, disintegrated, and/or metabolized slowly under physiological conditions following delivery (e.g., into a gastrointestinal tract of a subject, or following contact with gastric fluids or other bodily fluids). Useful sustained release materials are typically non-toxic and inert when contacted with fluids and tissues of mammalian subjects, and do not trigger significant adverse side effects such as irritation, immune response, inflammation, or the like. They are typically metabolized into metabolic products which are biocompatible and easily eliminated from the body.

In certain embodiments, sustained release polymeric materials are employed as the sustained release vehicle, matrix, binder, or coating (see, e.g., “Medical Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, N.Y. (1984); Ranger and Peppas, 1983, J Macromol. Sci. Rev. Macromol Chem. 23:61; see also Levy et al., 1985, Science 228: 190; During et al., 1989, Ann. Neurol. 25:351; Howard et al, 1989, J. Neurosurg. 71:105, each incorporated herein by reference). Within exemplary embodiments, useful polymers for co-formulating with the CNS therapeutic compound and/or lithium agent to yield a sustained release composition or dosage form include, but are not limited to, ethylcellulose, hydroxyethyl cellulose; hydroxyethylmethyl cellulose; hydroxypropyl cellulose; hydroxypropylmethyl cellulose; hydroxypropylmethyl cellulose phthalate; hydroxypropylmethylcellulose acetate succinate; hydroxypropylmethylcellulose acetate phthalate; sodium carboxymethylcellulose; cellulose acetate phthalate; cellulose acetate trimellitate; polyoxyethylene stearates; polyvinyl pyrrolidone; polyvinyl alcohol; copolymers of polyvinyl pyrrolidone and polyvinyl alcohol; polymethacrylate copolymers; and mixtures thereof.

Additional polymeric materials for use as sustained release vehicles, matrices, binders, or coatings within the compositions and dosage forms of the invention include, but are not limited to, additional cellulose ethers, e.g., as described in Alderman, Int. J. Pharm. Tech. & Prod. Mfr., 1984, 5(3) 1-9 (incorporated herein by reference). Other useful polymeric materials and matrices are derived from copolymeric and homopolymeric polyesters having hydrolysable ester linkages. A number of these are known in the art to be biodegradable and to lead to degradation products having no or low toxicity. Exemplary polymers in this context include polyglycolic acids (PGAs) and polylactic acids (PLAs), poly(DL-lactic acid-co-glycolic acid) (DL PLGA), poly(D-lactic acid-coglycolic acid) (D PLGA) and poly(L-lactic acid-co-glycolic acid) (L PLGA). Other biodegradable or bioerodable polymers for use within the invention include such polymers as poly(ε-caprolactone), poly(ε-aprolactone-CO-lactic acid), poly(ε-aprolactone-CO-glycolic acid), poly(β-hydroxy butyric acid), poly(alkyl-2-cyanoacrilate), hydrogels such as poly(hydroxyethyl methacrylate), polyamides, poly-amino acids (e.g., poly-L-leucine, poly-glutamic acid, poly-L-aspartic acid, and the like), poly (ester ureas), poly (2-hydroxyethyl DL-aspartamide), polyacetal polymers, polyorthoesters, polycarbonates, polymaleamides, polysaccharides, and copolymers thereof. Methods for preparing pharmaceutical formulations using these polymeric materials are generally known to those skilled in the art (see, e.g., Sustained and Controlled Release Drug Delivery Systems, J. R. Robinson, ed., Marcel Dekker, Inc., New York, 1978, incorporated herein by reference).

In other embodiments of the invention, the compositions and dosage forms comprise a CNS therapeutic compound and/or lithium agent coated on a polymer substrate. The polymer can be an erodible or a nonerodible polymer. The coated substrate may be folded onto itself to provide a bilayer polymer drug dosage form. For example the CNS therapeutic compound and/or lithium agent can be coated onto a polymer such as a polypeptide, collagen, gelatin, polyvinyl alcohol, polyorthoester, polyacetyl, or a polyorthocarbonate, and the coated polymer folded onto itself to provide a bilaminated dosage form. In operation, the bioerodible dosage form erodes at a controlled rate to dispense the active compound over a sustained release period. Representative biodegradable polymers for use in this and other aspects of the invention can be selected from, for example, biodegradable poly(amides), poly (amino acids), poly(esters), poly(lactic acid), poly(glycolic acid), poly(carbohydrate), poly(orthoester), poly (orthocarbonate), poly(acetyl), poly(anhydrides), biodegradable poly(dehydropyrans), and poly(dioxinones) which are known in the art (see, e.g., Rosoff, Controlled Release of Drugs, Chap. 2, pp. 53-95 (1989); and U.S. Pat. Nos. 3,811,444; 3,962,414; 4,066,747, 4,070,347; 4,079,038; and 4,093,709, each incorporated herein by reference).

In another embodiment of the invention, the dosage form comprises a CNS therapeutic compound and/or lithium agent loaded into a polymer that releases the drug(s) by diffusion through a polymer, or by flux through pores or by rupture of a polymer matrix. The drug delivery polymeric dosage form comprises the active compound contained in or on the polymer. The dosage form comprises at least one exposed surface at the beginning of dose delivery. The non-exposed surface, when present, can be coated with a pharmaceutically acceptable material impermeable to the passage of a drug. The dosage form may be manufactured by procedures known in the art, for example by blending a pharmaceutically acceptable carrier like polyethylene glycol, with a pre-determined dose of the active compound(s) at an elevated temperature (e.g., 37° C.), and adding it to a silastic medical grade elastomer with a cross-linking agent, for example, octanoate, followed by casting in a mold. The step is repeated for each optional successive layer. The system is allowed to set for 1 hour, to provide the dosage form. Representative polymers for manufacturing such sustained release dosage forms include, but are not limited to, olefin, and vinyl polymers, addition polymers, condensation polymers, carbohydrate polymers, and silicon polymers as represented by polyethylene, polypropylene, polyvinyl acetate, polymethylacrylate, polyisobutylmethacrylate, poly alginate, polyamide and polysilicon. These polymers and procedures for manufacturing them have been described in the art (see, e.g., Coleman et al., Polymers 1990, 31, 1187-1231; Roerdink et al., Drug Carrier Systems 1989, 9, 57-10; Leong et al., Adv. Drug Delivery Rev. 1987, 1, 199-233; and Roff et al., Handbook of Common Polymers 1971, CRC Press; U.S. Pat. No. 3,992,518, each of which is incorporated by reference herein in its entirety).

In other embodiments of the invention, the compositions and dosage forms comprise a CNS therapeutic compound and/or lithium agent incorporated with or contained in beads that on dissolution or diffusion release the active compound over an extended period of hours, for example over a period of at least 6 hours, over a period of at least 8 hours, over a period of at least 12 hours, or over a period of up to 24 hours or longer. The drug-releasing beads may have a central composition or core comprising an active compound of a CNS therapeutic compound and/or lithium agent and a pharmaceutically acceptable carrier, along with one or more optional excipients such as a lubricants, antioxidants, dispersants, and buffers. The beads may be medical preparations with a diameter of about 1 to 2 mm. In exemplary embodiments they are formed of non-cross-linked materials to enhance their discharge from the gastrointestinal tract. The beads may be coated with a release rate-controlling polymer that gives a timed release pharmacokinetic profile. In alternate embodiments the beads may be manufactured into a tablet for therapeutically effective drug administration. The beads can be made into matrix tablets by direct compression of a plurality of beads coated with, for example, an acrylic resin and blended with excipients such as hydroxypropylmethyl cellulose. The manufacture and processing of beads for use within the invention is described in the art (see, e.g., Lu, Int. J. Pharm., 1994, 112, 117-124; Pharmaceutical Sciences by Remington, 14^{th} ed, pp 1626-1628 (1970); Fincher, J. Pharm. Sci. 1968, 57, 1825-1835; and U.S. Pat. No. 4,083,949, each incorporated by reference) as has the manufacture of tablets (Pharmaceutical Sciences, by Remington, 17^{th} Ed, Ch. 90, pp 1603-1625, 1985, incorporated herein by reference).

In another embodiment of the invention, the dosage from comprises a plurality of tiny pills or mini-tablets. The tiny pills or mini-tablets provide a number of individual doses for providing various time doses for achieving a sustained-release drug delivery profile over an extended period of time up to 24 hours. The tiny pills or mini-tablets may comprise a hydrophilic polymer selected from the group consisting of a polysaccharide, agar, agarose, natural gum, alkali alginate including sodium alginate, carrageenan, fucoidan, furcellaran, laminaran, hypnea, gum arabic, gum ghatti, gum karaya, grum tragacanth, locust bean gum, pectin, amylopectin, gelatin, and a hydrophilic colloid. The hydrophilic polymer may be formed into a plurality (e.g., 4 to 50) tiny pills or mini-tablet, wherein each tiny pill or mini-tablet comprises a pre-determined dose of the CNS therapeutic compound and/or lithium agent, e.g., a dose of about 10 ng, 0.5 mg, 1 mg, 1.2 mg, 1.4 mg, 1.6 mg, 5.0 mg etc. The tiny pills and mini-tablets may further comprise a release rate-controlling wall of 0.001 up to 10 mm thickness to provide for timed release of the active compound. Representative wall forming materials include a triglyceryl ester selected from the group consisting of glyceryl tristearate, glyceryl monostearate, glyceryl dipalmitate, glyceryl laureate, glyceryl didecenoate and glyceryl tridenoate. Other wall forming materials comprise polyvinyl acetate, phthalate, methylcellulose phthalate and microporous olefins. Procedures for manufacturing tiny pills and mini-tablets are known in the art (see, e.g., U.S. Pat. Nos. 4,434,153; 4,721,613; 4,853,229; 2,996,431; 3,139,383 and 4,752,470, each incorporated herein by reference). The tiny pills and mini-tablets may further comprise a blend of particles, which may include particles of different sizes and/or release properties, and the particles may be contained in a hard gelatin or non-gelatin capsule or soft gelatin capsule.

In yet another embodiment of the invention, drug-releasing lipid matrices can be used to formulate therapeutic compositions and dosage forms comprising a CNS therapeutic compound and/or lithium agent. In one exemplary embodiment, solid microparticles of the active compound are coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700 (each incorporated herein by reference in its entirety). The lipid-coated particles can optionally be compressed to form a tablet. Another controlled release lipid-based matrix material which is suitable for use in the sustained release compositions and dosage forms of the invention comprises polyglycolized glycerides, e.g., as described in Roussin et al., U.S. Pat. No. 6,171,615 (incorporated herein by reference in its entirety).

In other embodiments of the invention, drug-releasing waxes can be used for producing sustained release compositions and dosage forms comprising a CNS therapeutic compound and/or lithium agent. Examples of suitable sustained drug-releasing waxes include, but are not limited to, carnauba wax, candedilla wax, esparto wax, ouricury wax, hydrogenated vegetable oil, bees wax, paraffin, ozokerite, castor wax, and mixtures thereof (see, e.g., Cain et al., U.S. Pat. No. 3,402,240; Shtohryn et al., U.S. Pat. No. 4,820,523; and Walters, U.S. Pat. No. 4,421,736, each incorporated herein by reference in its entirety).

In still another embodiment, osmotic delivery systems are used for sustained release delivery of a CNS therapeutic compound and/or lithium agent (see, e.g., Verma et al., Drug Dev. Ind. Pharm., 2000, 26:695-708, incorporated herein by reference). In one exemplary embodiment, the osmotic delivery system is an OROS® system (Alza Corporation, Mountain View, Calif.) and is adapted for oral sustained release delivery of drugs (see, e.g., U.S. Pat. No. 3,845,770; and U.S. Pat. No. 3,916,899, each incorporated herein by reference in its entirety).

In another embodiment of the invention, the dosage form comprises an osmotic dosage form, which comprises a semi-permeable wall that surrounds a therapeutic composition comprising the CNS therapeutic compound and/or lithium agent. In use within a patient, the osmotic dosage form comprising a homogenous composition imbibes fluid through the semi-permeable wall into the dosage form in response to the concentration gradient across the semi-permeable wall. The therapeutic composition in the dosage form develops osmotic energy that causes the therapeutic composition to be administered through an exit from the dosage form over a prolonged period of time up to 24 hours (or even in some cases up to 30 hours) to provide controlled and sustained prodrug release. These delivery platforms can provide an essentially zero order delivery profile as opposed to the spiked profiles of immediate release formulations.

In alternate embodiments of the invention, the dosage form comprises another osmotic dosage form comprising a wall surrounding a compartment, the wall comprising a semi-permeable polymeric composition permeable to the passage of fluid and substantially impermeable to the passage of the active compound present in the compartment, a drug-containing layer composition in the compartment, a hydrogel push layer composition in the compartment comprising an osmotic formulation for imbibing and absorbing fluid for expanding in size for pushing the CNS therapeutic compound and/or lithium agent composition layer from the dosage form, and at least one passageway in the wall for releasing the drug composition. This osmotic system delivers the active compound by imbibing fluid through the semi-permeable wall at a fluid imbibing rate determined by the permeability of the semi-permeable wall and the osmotic pressure across the semi-permeable wall causing the push layer to expand, thereby delivering the active compound through the exit passageway to a patient over a prolonged period of time (up to 24 or even 30 hours). The hydrogel layer composition may comprise 10 mg to 1000 mg of a hydrogel such as a member selected from the group consisting of a polyalkylene oxide of 1,000,000 to 8,000,000 which are selected from the group consisting of a polyethylene oxide of 1,000,000 weight-average molecular weight, a polyethylene oxide of 2,000,000 molecular weight, a polyethylene oxide of 4,000,000 molecular weight, a polyethylene oxide of 5,000,000 molecular weight, a polyethylene oxide of 7,000,000 molecular weight and a polypropylene oxide of the 1,000,000 to 8,000,000 weight-average molecular weight; or 10 mg to 1000 mg of an alkali carboxymethylcellulose of 10,000 to 6,000,000 weight average molecular weight, such as sodium carboxymethylcellulose or potassium carboxymethylcellulose. The hydrogel expansion layer may comprise a hydroxyalkylcellulose of 7,500 to 4,500,00 weight-average molecular weight (e.g., hydroxymethylcellulose, hydroxyethylcellulose, hydroxypropylcellulose, hydroxybutylcellulose or hydroxypentylcellulose), an osmagent, e.g., selected from the group consisting of sodium chloride, potassium chloride, potassium acid phosphate, tartaric acid, citric acid, raffinose, magnesium sulfate, magnesium chloride, urea, inositol, sucrose, glucose and sorbitol, and other agents such a hydroxypropylalkylcellulose of 9,000 to 225,000 average-number molecular weight (e.g., hydroxypropylethylcellulose, hydroxypropypentylcellulose, hydroxypropylmethylcellulose, or hydropropylbutylcellulose), ferric oxide, antioxidants (e.g., ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin and ethanolamine), and/or lubricants (e.g., calcium stearate, magnesium stearate, zinc stearate, magnesium oleate, calcium palmitate, sodium suberate, potassium laureate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid).

In the osmotic dosage forms, the semi-permeable wall comprises a composition that is permeable to the passage of fluid and impermeable to passage of the CNS therapeutic compound and/or lithium agent. The wall is nontoxic and comprises a polymer selected from the group consisting of a cellulose acylate, cellulose diacylate, cellulose triacylate, cellulose acetate, cellulose diacetate and cellulose triacetate. The wall typically comprises 75 wt % (weight percent) to 100 wt % of the cellulosic wall-forming polymer; or, the wall can comprise additionally 0.01 wt % to 80 wt % of polyethylene glycol, or 1 wt % to 25 wt % of a cellulose ether (e.g., hydroxypropylcellulose or a hydroxypropylalkycellulose such as hydroxypropylmethylcellulose). The total weight percent of all components comprising the wall is equal to 100 wt %. The internal compartment comprises the drug-containing composition alone or in layered position with an expandable hydrogel composition. The expandable hydrogel composition in the compartment increases in dimension by imbibing the fluid through the semi-permeable wall, causing the hydrogel to expand and occupy space in the compartment, whereby the drug composition is pushed from the dosage form. The therapeutic layer and the expandable layer act together during the operation of the dosage form for the release of drug to a patient over time. The dosage form comprises a passageway in the wall that connects the exterior of the dosage form with the internal compartment. The osmotic powered dosage form delivers the CNS therapeutic compound and/or lithium agent from the dosage form to the patient at a zero order rate of release over a period of up to about 24 hours. As used herein, the expression “passageway” comprises means and methods suitable for the metered release of a CNS therapeutic compound and/or lithium agent from the compartment of an osmotic dosage form. The exit means comprises at least one passageway, including orifice, bore, aperture, pore, porous element, hollow fiber, capillary tube, channel, porous overlay, or porous element that provides for the osmotic controlled release of the active compound. The passageway includes a material that erodes or is leached from the wall in a fluid environment of use to produce at least one controlled-release dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of prodrug from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in U.S. Pat. Nos. 3,845,770; 3,916,899; 4,063,064; 4,088,864; 4,816,263; 4,200,098; and 4,285,987 (each incorporated herein by reference in its entirety).

Within other aspects of the invention, microparticle, microcapsule, and/or microsphere drug delivery technologies can be employed to provide sustained release delivery of a CNS therapeutic compound and/or lithium agent within the compositions, dosage forms and methods of the invention. A variety of methods is known by which an CNS therapeutic compound and/or lithium agent can be encapsulated in the form of microparticles, for example using by encapsulating the active compound within a biocompatible, biodegradable wall-forming material (e.g., a polymer)—to provide sustained or delayed release of the active compound. In these methods, the active compound is typically dissolved, dispersed, or emulsified in a solvent containing the wall forming material. Solvent is then removed from the microparticles to form the finished microparticle product. Examples of conventional microencapsulation processes are disclosed, e.g., in U.S. Pat. Nos. 3,737,337; 4,389,330; 4,652,441; 4,917,893; 4,677,191; 4,728,721; 5,407,609; 5,650,173; 5,654,008; and 6,544,559 (each incorporated herein by reference in their entirety). These documents disclose methods that can be readily implemented to prepare microparticles containing a CNS therapeutic compound and/or lithium agent in a sustained release formulation according to the invention. As explained, for example, in U.S. Pat. No. 5,650,173, by appropriately selecting the polymeric materials, a microparticle formulation can be made in which the resulting microparticles exhibit both diffusional release and biodegradation release properties. For a diffusional mechanism of release, the active agent is released from the microparticles prior to substantial degradation of the polymer. The active agent can also be released from the microparticles as the polymeric excipient erodes. In addition, U.S. Pat. No. 6,596,316 (incorporated herein by reference) discloses methods for preparing microparticles having a selected release profile for fine tuning a release profile of an active agent from the microparticles.

In another embodiment of the invention, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release). Enteric coatings may function as a means for mediating sustained release of the CNS therapeutic agent and/or lithium agent by providing one or more barrier layers, which may be located entirely surrounding the active compound, between layers of a multi-layer solid dosage form (see below), and/or on one or more outer surfaces of one or multiple layers of a multi-layer solid dosage form (e.g., on end faces of layers of a substantially cylindrical tablet). Such barrier layers may, for example, be composed of polymers which are either substantially or completely impermeable to water or aqueous media, or are slowly erodible in water or aqueous media or biological liquids and/or which swell in contact with water or aqueous media. Suitable polymers for use as a barrier layer include acrylates, methacrylates, copolymers of acrylic acid, celluloses and derivatives thereof such as ethylcelluloses, cellulose acetate propionate, polyethylenes and polyvinyl alcohols etc. Barrier layers comprising polymers which swell in contact with water or aqueous media may swell to such an extent that the swollen layer forms a relatively large swollen mass, the size of which delays its immediate discharge from the stomach into the intestine. The barrier layer may itself contain active material content, for example the barrier layer may be a slow or delayed release layer. Barrier layers may typically have an individual thickness of 10 microns up to 2 mm. Suitable polymers for barrier layers which are relatively impermeable to water include the Methocel™ series of polymers, used singly or combined, and Ethocel™ polymers. Such polymers may suitably be used in combination with a plasticizer such as hydrogenated castor oil. The barrier layer may also include conventional binders, fillers, lubricants and compression acids etc such as Polyvidon K30 (trade mark), magnesium stearate, and silicon dioxide.

Additional enteric coating materials for mediating sustained release of a CNS therapeutic compound and/or lithium agent include coatings in the form of polymeric membranes, which may be semi-permeable, porous, or asymmetric membranes (see, e.g., U.S. Pat. No. 6,706,283, incorporated herein by reference). Coatings of these and other types for use within the invention may also comprise at least one delivery port, or pores, in the coating, e.g., formed by laser drilling or erosion of a plug of water-soluble material. Other useful coatings within the invention including coatings that rupture in an environment of use (e.g., a gastrointestinal compartment) to form a site of release or delivery port. Exemplary coatings within these and other embodiments of the invention include poly(acrylic) acids and esters; poly(methacrylic) acids and esters; copolymers of poly(acrylic) and poly(methacrylic) acids and esters; cellulose esters; cellulose ethers; and cellulose ester/ethers.

Additional coating materials for use in constructing solid dosage forms to mediate sustained release of a CNS therapeutic compound and/or lithium agent include, but are not limited to, polyethylene glycol, polypropylene glycol, copolymers of polyethylene glycol and polypropylene glycol, poly(vinylpyrrolidone), ethyl cellulose, hydroxyethyl cellulose, hydroxypropyl cellulose, carboxymethyl cellulose, carboxymethylethyl cellulose, starch, dextran, dextrin, chitosan, collagen, gelatin, bromelain, cellulose acetate, unplasticized cellulose acetate, plasticized cellulose acetate, reinforced cellulose acetate, cellulose acetate phthalate, cellulose acetate trimellitate, hydroxypropylmethylcellulose, hydroxypropylmelhyl-cellulose phthalate, hydroxypropylmethylcellulose acetate succinate, hydroxypropylmethylcellulose acetate trimellitate, cellulose nitrate, cellulose diacetate, cellulose triacetate, agar acetate, amylose triacetate, beta glucan acetate, beta glucan triacetate, acetaldehyde dimethyl acetate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, cellulose acetate methyl sulfonate, cellulose acetate butyl sulfonate, cellulose acetate propionate, cellulose acetate p-toluene sulfonate, triacetate of locust gum bean, cellulose acetate with acetylated hydroxyethyl cellulose, hydroxlated ethylene-vinylacetate, cellulose acetate butyrate, polyalkenes, polyethers, polysulfones, polyethersulfones, polystyrenes, polyvinyl halides, polyvinyl esters and ethers, natural waxes and synthetic waxes.

In additional embodiments of the invention, sustained release of the CNS therapeutic compound and/or lithium agent is provided by formulating the active compound in a dosage form comprising a multi-layer tablet or other multi-layer or multi-component dosage form. In exemplary embodiments, the active compound is formulated in layered tablets, for example having a first layer which is an immediate release layer and a second layer which is a slow release layer. Other multi-layered dosage forms of the invention may comprise a plurality of layers of compressed active ingredient having variable (i.e., selectable) release properties selected from immediate, extended and/or delayed release mechanisms. Multi-layered tablet technologies useful to produce sustained release dosage forms of a CNS therapeutic agent and/or a lithium agent are described, for example, in International Publications WO 95/20946; WO 94/06416; and WO 98/05305 (each incorporated herein by reference in its entirety). Other multi-component dosage forms for providing sustained delivery of a CNS therapeutic compound and/or lithium agent include tablet formulations having a core containing the active compound coated with a release retarding agent and surrounded by an outer casing layer (optionally containing the active compound) (see, e.g., International Publication WO 95/28148, incorporated herein by reference in its entirety). The release retarding agent is an enteric coating, so that there is an immediate release of the contents of the outer core, followed by a second phase from the core which is delayed until the core reaches the intestine. Additionally, International Publication WO 96/04908 (incorporated herein by reference in its entirety) describes tablet formulations which comprise an active agent in a matrix, for immediate release, and granules in a delayed release form comprising the active agent. Such granules are coated with an enteric coating, so release is delayed until the granules reach the intestine. International Publication WO 96/04908 (incorporated herein by reference) describes delayed or sustained release formulations formed from granules which have a core comprising an active agent, surrounded by a layer comprising the active agent.

Another useful multi-component (bi-layer tablet) dosage form for sustained delivery of a CNS therapeutic agent and/or a lithium agent is described in U.S. Pat. No. 6,878,386 (incorporated herein by reference in its entirety). Briefly, the bilayer tablet comprises an immediate release and a slow release layer, optionally with a coating layer. The immediate release layer may be, for example, a layer which disintegrates immediately or rapidly and has a composition similar to that of known tablets which disintegrate immediately or rapidly. An alternative type of immediate release layer may be a swellable layer having a composition which incorporates polymeric materials which swell immediately and extensively in contact with water or aqueous media, to form a water permeable but relatively large swollen mass. Active material content may be immediately leached out of this mass. The slow release layer may have a composition comprising the CNS therapeutic compound and/or lithium agent with a release retarding vehicle, matrix, binder, coating, or excipient which allows for slow release of the active compound. Suitable release retarding excipients include pH sensitive polymers, for instance polymers based upon methacrylic acid copolymers, which may be used either alone or with a plasticizer; release-retarding polymers which have a high degree of swelling in contact with water or aqueous media such as the stomach contents; polymeric materials which form a gel on contact with water or aqueous media; and polymeric materials which have both swelling and gelling characteristics in contact with water or aqueous media. Release retarding polymers which have a high degree of swelling include, inter alia, cross-linked sodium carboxymethylcellulose, cross-linked hydroxypropylcellulose, high-molecular weight hydroxypropylmethylcellulose, carboxymethylamide, potassium methacrylatedivinylbenzene co-polymer, polymethylmethacrylate, cross-linked polyvinylpyrrolidone, high-molecular weight polyvinylalcohols etc. Release retarding gellable polymers include, but are not limited to, methylcellulose, carboxymethylcellulose, low-molecular weight hydroxypropylmethylcellulose, low-molecular weight polyvinylalcohols, polyoxyethyleneglycols, non-cross linked polyvinylpyrrolidone, xanthan gum etc. Release retarding polymers simultaneously possessing swelling and gelling properties include medium-viscosity hydroxypropylmethylcellulose and medium-viscosity polyvinylalcohols. An exemplary release-retarding polymer is xanthan gum, in particular a fine mesh grade of xanthan gum, preferably pharmaceutical grade xanthan gum, 200 mesh, for instance the product Xantural 75 (also known as Keltrol CR™ Monsanto, 800 N Lindbergh Blvd, St Louis, Mo. 63167, USA). Xanthan gum is a polysaccharide which upon hydration forms a viscous gel layer around the tablet through which the active has to diffuse. It has been shown that the smaller the particle size, the slower the release rate. In addition, the rate of release of active compound is dependent upon the amount of xanthan gum used and can be adjusted to give the desired profile. Examples of other polymers which may be used within these aspects of the invention include Methocel K4M™, Methocel E5™, Methocel E5O™, Methocel E4M™, Methocel K15M™ and Methocel K100M™. Other known release-retarding polymers which may be incorporated within this and other embodiments of the invention to provide a sustained release composition or dosage form of a CNS therapeutic compound and/or lithium agent include, hydrocolloids such as natural or synthetic gums, cellulose derivatives other than those listed above, carbohydrate-based substances such as acacia, gum tragacanth, locust bean gum, guar gum, agar, pectin, carageenan, soluble and insoluble alginates, carboxypolymethylene, casein, zein, and the like, and proteinaceous substances such as gelatin.

Within other embodiments of the invention, a sustained release delivery device or system is placed in the subject in proximity of the target of the active compound, thus requiring only a fraction of the systemic dose (see, e.g., Goodson, in “Medical Applications of Controlled Release,” supra, vol. 2, pp. 115-138, 1984; and Langer, 1990, Science 249:1527-1533, each incorporated herein by reference in its entirety). In other embodiments, an oral sustained release pump may be used (see, e.g., Langer, supra; Sefton, 1987, CRC Crit. Ref. Biomed. Eng. 14:201; and Saudek et al., 1989, N. Engl. J. Med. 321:574, each incorporated herein by reference in its entirety).

The pharmaceutical compositions and dosage forms of the current invention will typically be provided for administration in a sterile or readily sterilizable, biologically inert, and easily administered form.

In other embodiments the invention provides pharmaceutical kits for reducing suicidality in a human subject suffering from a central nervous system (CNS) disorder. The kits comprise a CNS therapeutic agent in an amount effective to treat the targeted CNS disorder, lithium in an amount effective to reduce suicidality in the subject, and a container means for containing the CNS therapeutic agent and said lithium for coordinate administration to the said subject (for example a container, divided bottle, or divided foil pack). The container means can include a package bearing a label or insert that provides instructions for multiple uses of the kit contents to treat the CNS disorder and reduce suicidality in the subject. In more detailed embodiments, the CNS therapeutic agent and lithium are admixed or co-formulated in a single, combined dosage form, for example a liquid or solid oral dosage form. In alternate embodiments, the CNS therapeutic agent and lithium are contained in the kit in separate dosage forms for coordinate administration. An example of such a kit is a so-called blister pack. Blister packs are well-known in the packaging industry and are widely used for the packaging of pharmaceutical dosage forms (tablets, capsules and the like). Blister packs generally consist of a sheet of relatively stiff material covered with a foil of a transparent material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil that is opposite from the direction in which the recesses were formed. As a result, the tablets or capsules are sealed in the recesses between the plastic foil and the sheet. Preferably, the strength of the sheet is such that the tablets or capsules can be removed from the blister pack by manually applying pressure on the recesses whereby an opening is formed in the sheet at the place of the recess. The tablet or capsule can then be removed from the opening. Often, a memory aid is provided on the kit, such as in the form of numbers next to the tablets or capsules whereby the numbers correspond with the days of the regimen which the tablets or capsules so specified should be ingested. Other variations of memory aids will be readily apparent.

The following examples illustrate certain embodiments of the present invention, and are not to be construed as limiting the present disclosure.

### Example I

### Efficacy of Combination Lithium/Citalopram Treatment for Reducing Suicidality in Patients Presenting with Depression and Current Suicidal Ideation or Having a History of Suicidal Ideation

Prior studies we have conducted over a lengthy course of clinical investigation have revealed a surprising activity of lithium for reducing suicidality of patients with various forms of depressive disorders, including major depression taking anti-depressant drugs to treat the underlying depressive disorder. In particular, we have observed that in patients with depressive disorders taking anti-depressant drugs in a standard therapeutic dose and treatment regiment, the addition of lithium to patients' treatment regimens substantially reduced the incidence of suicide and suicidal behavior and ideation. Even at lower than expected therapeutic dosages, e.g., below about 1,800-2,000 per day, even below about 500 mg/day, 300 mg/day, or less, lithium administered coordinately with the subject anti-depressant drug yielded a substantially reduced rate of suicide, suicidal behavior, and suicidal ideation. In exemplary studies, we have observed that lithium coordinately administered with various anti-depressants, including SSRI anti-depressants, for example, citalopram, was anti-suicidally effective to yield surprising reductions in patient indices of suicidality, e.g., as measured by one or more of the suicidality scales identified above. These observed reductions in suicidality have led us to conclude that test subjects administered lithium in combination with a standard anti-depressant, such as an SSRI anti-depressant, will exhibit a significant reduction in one or more scores or selected values generated from exemplary suicidality determining surveys or scales as described above (indicating for example an incidence or severity of a suicidal behavior, condition, or ideation), by at least 5%, 10%, 20%, 30%, 50% or greater, up to a 75-90%, or 95% compared to correlative scores or values observed for control subjects treated with placebo or other suitable control treatment. At the same time, patients treated with a combination of lithium and an anti-depressant drug are expected to exhibit not only a marked reduction (especially at low lithium dosages of, for example, 200-500 mg/day) in one or more target symptom(s) associated with the subject depressive disorder under investigation, for example major depression, by at least 5%, 10%, 20%, 30%, 50% or greater, up to a 75-90%, or 95% or greater, compared to patient indices/symptoms of the subject depressive disorder in placebo-treated or other suitable control subjects. As demonstrated below, administration of a combination of lithium and citalopram yielded a reduction in suicidal thoughts, desires and self-harming behaviors.

### Phase II Clinical Investigation

Ninety-three patients were screened and 80 subjects were identified who were between the ages of 18 to 75, inclusive, and met the criteria for major depressive disorder (MDD), dysthymia, depression not otherwise specified and/or borderline personality disorder as defined by Diagnostic and Statistical Manual-IV-TR and confirmed by the Mini International Neuropsychological Interview (MINI).

Selected subjects scored >15 on the Montgomery-Åsberg Depression Rating Scale and >8 on the Sheehan Suicidality Tracking Scale (S-STS) at Screening and Baseline Visits. Subjects were excluded if they had current, unstable and significant medical conditions (a history of unstable Thyroid disorders, a history of seizure disorders, tumors or other central nervous system (CNS) condition that predisposes the patient to risk of seizure), pregnant or lactating, had any abnormal clinical laboratory test results, or ECG results which were judged by the investigator as clinically significant, a history of intolerance or hypersensitivity to citalopram, other SSRIs, or lithium, a history or current diagnosis of bipolar mood disorder, psychosis, schizophrenia or dementia, were currently involved in another clinical trial or had participated in another clinical trial less than thirty days prior, or had current or prior treatment with citalopram or escitalopram. Psychotropic medications (anti-psychotics, anti-depressants, and mood stabilizers) were excluded during the treatment phase and required a twenty-four hour washout period prior to the first dose of study medication. Diuretics, analgesics, benzodiazepines and opiates were used with caution.

The mean age of the study participants was 41 (±14). There were 44 female participants and 36 male participants. 68% of the participants were white, 21% black, 5% Asian, 3% Pacific Island, and 3% other.

Eligible subjects were evaluated for safety parameters prior to and throughout the screening through a variety of methods including an ECG, physical exam, vital sign taking and clinical laboratory testing. A standard 12-lead ECG was performed at Screening (Visit 1) and at Week 4 (Visit 6 or Early Termination Visit). Physical examinations included determination of general appearance, mental status, skin, neck (including thyroid), eyes, ears, nose, throat, heart, lungs, abdomen, lymph nodes, musculoskeletal system, and nervous system. Vital signs included respiratory rate, radial pulse and systolic and diastolic blood pressures. Vital signs were collected in the seated position. Clinical Laboratory Testing Parameters included a Complete Metabolic Panel (Na, K, Cl, CO2, Glu, BUN, Cr, Ca, TP, Alb, TBili, AP, AST, ALT), Hematology CBC (Hgb, Hct, RBC, WBC, Plt, Diff) and TSH (w/reflex T-4). Serum HCG is performed for all females, and Urine Analysis is completed for all subjects.

Eligible subjects not eliminated by the safety parameters were divided into two groups of forty as shown in Table 4. All subjects received 20 mg citalopram and were randomized to either 300 mg lithium carbonate/day or placebo for 4 weeks of Double-Blind Treatment. Patients were instructed to take one capsule from the bottle and two tablets from the blister card one time daily, in the morning, orally with food. If the patient was unable to tolerate morning dosing, patient may be switched to evening dosing as long as there was 24 hours minimum between doses.

TABLE 4
Characteristics of eighty severely depressed patients
with suicidal thoughts and behaviors assigned to citalopram +
placebo and citalopram + lithium trial arms.

| | Citalopram + | Citalopram + |
|---|---|---|
| | Placebo | Lithium |
| | n = 40 | n = 40 |
| Mean Age | 38.5 | 45.0 |
| % Female | 62.5 | 47.5 |
| % Caucasian | 62.5 | 72.5 |
| % with Borderline Personality Disorder | 42.5 | 40.0 |
| % with Prior Suicide Attempt(s) | 65.0 | 67.5 |
| % with Prior Suicide Attempt(s) within | 10.0 | 2.5 |
| the Past Month | | |
| % with Prior Suicide Attempt(s) within | 17.5 | 15.0 |
| the Past Year | | |
| % with History of Self-Harm Behavior | 27.5 | 15.0 |
| % Taking any Concomitant Medication(s) | 57.5 | 77.5 |

Patients were evaluated according to the following schedule of events:

TABLE 5
Schedule of Events

| | Visit 1/ | Visit 2/ | Visit 3/ | Visit 4/ | Visit 5/ | Visit 6/ | 1 week |
|---|---|---|---|---|---|---|---|
| Procedure | Screen | Baseline | Week 1 | Week 2 | Week 3 | Wk 4 or ET | Follow-up |
| Informed Consent | X | | | | | | |
| Patient Demographics | X | | | | | | |
| Vital Signs | X | X | X | X | X | X | |
| Height | X | | | | | | |
| Weight | X | X | X | X | X | X | |
| 12-lead ECG | X | | | | | X | |
| Physical Examination | X | | | | | X | |
| Medical History | X | | | | | | |
| Psychiatric History | X | | | | | | |
| Diagnosis | X | | | | | | |
| Inclusion/Exclusion | X | X | | | | | |
| Criteria | | | | | | | |
| Lab Work | X | | | | | X | |
| Blood Draw to measure | | | | | | X | |
| lithium levels | | | | | | | |
| Urine Drug Screen | X | | | | | | |
| Serum Pregnancy Test | X | | | | | X | |
| (females only) | | | | | | | |
| MINI | X | | | | | | |
| Sheehan Suicidality | X | X | X | X | X | X | |
| Tracking Scale (S-STS) | | | | | | | |
| Beck Hopelessness | X | X | X | X | X | X | |
| Scale | | | | | | | |
| Beck Scale for Suicide | X | X | X | X | X | X | |
| Ideation | | | | | | | |
| MADRS | X | X | X | X | X | X | |
| (Montgomery-Åsberg | | | | | | | |
| Depression Rating | | | | | | | |
| Scale (Montgomery | | | | | | | |
| and Åsberg, 1979)) | | | | | | | |
| CGI | X | X | X | X | X | X | |
| C-SSRS | X | | | | | X | |
| Adverse Events | | X | X | X | X | X | X |
| Medication Dispensed | | X | X | X | X | | |
| Phone Call | | | | | | | X |

Efficacy was measured by taking S-STS (Sheehan-Suicidality Tracking Scale (Sheehan, 2009)) scores at Baseline and at each visit for 4 weeks of treatment or until end-of-treatment and measuring the change from baseline in the S-STS total score at week 4. Secondary efficacy variables were the BHS (Beck Hopelessness Scale (Beck, Steer, 1988)), BSS (Beck Scale for Suicide Ideation (Beck and Steer, 1991)), C-SSRS (Columbia Suicide Severity Rating Scale (Posner et al., 2009) and CGI (Clinical Global Impression Study (Guy, 1976)) changes at week 4. Safety measures were collected and evaluated throughout the trial. These included Adverse Events (AEs), laboratory parameters, physical examinations, ECGs and vital signs and weight measurements. Adverse events were any untoward medical event occurring in a subject administered study drug, irrespective of whether it has a causal relationship to the study drug. An adverse event could therefore be any unfavorable or unintended sign (including abnormal laboratory findings, for example), symptom, or disorder temporarily associated with study drug, whether or not considered related to the study drug. Adverse Events among the 80 participants were as follows:

TABLE 6
Most Frequent and Serious Adverse Events

| | Citalopram + | Citalopram + | |
|---|---|---|---|
| Adverse Event | Placebo | Lithium | Total |
| Somnolence | 7 (18%) | 6 (15%) | 13 (16%) |
| Nausea | 5 (3%) | 6 (15%) | 11 (14%) |
| Headaches | 1 (1%) | 9 (23%) | 10 (13%) |
| Dizziness | 2 (5%) | 2 (5%) | 4 (5%) |
| Vomiting | 1 (3%) | 2 (5%) | 3 (4%) |
| Decreased Appetite | 2 (5%) | 2 (5%) | 4 (5%) |
| Anxiousness | 1 (3%) | 2 (5%) | 3 (4%) |
| Restlessness | 2 (5%) | — | 2 (3%) |
| Common Cold | 2 (5%) | 2 (5%) | 4 (5%) |
| Total Events | 23 | 31 | 54 |

These Adverse Events are consistent with the known drug effects mentioned in the package inserts for citalopram and lithium.

All significance tests were two-sided. Statistical significance was assessed at the p≦0.05 level for main effects, and significance at the p≦0.10 level was considered a trend indicating efficacy. Missing values were handled using the last observation carried forward (LOCF) technique. The primary analysis population for inference was the intention-to-treat (ITT) population.

Subjects were considered to have completed the study if they completed all of the visits. They were terminated from the study if they failed to meet inclusion/exclusion criteria; suffered from an adverse event, had an insufficient therapeutic response, withdrew their consent, violated the protocol, stopped coming, or died. Of the 80 participants in the study, 64 patients completed the study as planned and 16 patients discontinued participation. (1 Adverse Event (Citalopram+Placebo group), 7 out of Visit Window (4 Citalopram+Placebo group, 3 Citalopram+Lithium group), 3 Moved out of State (1 Citalopram+Placebo group. 2 Citalopram+Lithium group), 5 withdrew consent (2 Citalopram+Placebo group, 3 Citalopram+Lithium group)).

As shown in Table 7, there were no significant differences in the reduction of suicidal thoughts or behaviors between the two groups of depressed outpatients.

TABLE 7
Mean (SD) Baseline and End of Study Efficacy Outcomes for Patients Assigned
to Citalopram + Placebo Versus Citalopram + Lithium

| | Citalopram + Placebo | | | Citalopram + Lithium | | |
|---|---|---|---|---|---|---|
| | | End of | | | End of | |
| Assessment | Baseline | Study | F | Baseline | Study | F |
| S-STS | 12.0 (3.7) | 7.2 (5.5) | 13.0* | 10.8 (2.3) | 5.8 (5.1) | 12.4* |
| MADRS | 37.7 (4.8) | 27.8 (11.1) | 7.9* | 37.2 (3.1) | 26.5 (11.6) | 8.6* |
| CGI (Severity) | 4.9 (0.8) | 3.3 (1.8) | 11.9* | 4.7 (0.7) | 2.9 (1.7) | 14.1* |
| BSS | 16.2 (6.7) | 9.1 (8.2) | 11.7* | 13.7 (6.9) | 7.9 (7.9) | 8.3* |
| BHS | 16.3 (3.4) | 11.8 (5.4) | 6.1* | 15.5 (4.6) | 12.0 (6.3) | 3.3** |

Note.
F value derived from Repeated Measures Analysis of Variance assessing improvement over time within each treatment group.
*p ≦ 0.001,
**p < 0.05

However, the amount of change differed based on the baseline levels of suicidality, (F=10.0, p=0.002) with those patients that had a higher level of suicidality at baseline experiencing greater change scores. As shown in Table 7 above, the change in suicide risk measures such as S-STS and BSS was twice as large as changes in the depressive symptoms measured by MADRS and BHS. S-STS decreased 25% in the first week of trial participation.

In addition, as shown in Table 8, below, the subgroup of depressed patients (n=11) with a serum lithium level that was at the clinically therapeutic level of 0.5 mEq/L or higher (3 at 0.5 mEq/L, 3 at 0.6 mEq/L, and 5 at 0.7 mEq/L) had several indicators for better outcome on suicide risk measures.

TABLE 8
Efficacy and Safety Measures Between Patients Assigned to Citalopram + Placebo
and Patients Treated With Citalopram + Lithium Based on Serum Lithium Levels

| | | | Citalopram + Lithium | | Citalopram + Lithium | |
|---|---|---|---|---|---|---|
| Outcome | Citalopram + Placebo | | (Non-therapeutic Level) | | (Therapeutic Level) | |
| Measure | n = 40 | | n = 29 | | n = 11 | |
| Mean (SD) Total Baseline | 12.0 | (3.7) | 10.6 | (2.3) | 11.5 | (2.4) |
| S-STS Score | | | | | | |
| Mean (SD) Total S-STS Score | 7.2 | (5.5) | 6.1 | (4.9) | 4.7 | (5.6) |
| at the End of Trial | | | | | | |
| Mean (SD) Total S-STS | 4.8 | (5.1) | 4.4 | (4.9) | 6.7 | (5.8) |
| Change Score | | | | | | |
| Mean (SD) Total Baseline | 37.7 | (4.9) | 37.1 | (2.8) | 37.4 | (3.8) |
| MADRS Score | | | | | | |
| Mean (SD) Total MADRS | 27.8 | (11.1) | 26.8 | (11.5) | 25.6 | (12.3) |
| Score at the End of Trial | | | | | | |
| Mean (SD) Total MADRS | 9.8 | (11.4) | 10.3 | (11.7) | 11.8 | (14.4) |
| Change Score | | | | | | |
| # With Full S-STS Symptom | 6 | (15%) | 7 | (24%) | 5 | (45%)* |
| Remission | | | | | | |
| # With No change in | 12 | (30%) | 8 | (28%) | 3 | (27%) |
| S-STS Symptoms | | | | | | |

| | | | | | |
|---|---|---|---|---|---|
| # with Worsening of S-STS | 2 | (5%) | 3 | (10.3%) | 0 |
| Symptoms | | | | | |
| # Early Terminations | 8 | (20%) | 8 | (28%) | 0** |

| | | | |
|---|---|---|---|
| Treatment Compliance | 78.7%*** | 98.0% | 99.6% |
| Adverse Events per Patient | 1.0 | 1.3 | 1.3 |

Note.
S-STS = Sheehan Suicidality Tracking Scale, MADRS = Montgomery-Åsberg Depression Rating Scale, BSS = Beck Suicide Scale, BHS = Beck Hopelessness Scale.
*Therapeutic Serum Lithium versus all others χ² = 3.9, p = 0.049,
**Therapeutic Serum Lithium versus all others χ² = 3.2, p = 0.074.
***Four patients in the citalopram + placebo group had 0% compliance. With the exception of these patients the compliance between all treatment arms was similar.

Five of these 11 patients (45%) had a complete remission in suicidal thoughts, desires and self-harm behaviors compared to 13 of the rest of the 69 patients (18.8%, χ²=3.9, p=0.049) and none experienced worsening of symptoms. Further, none of these 11 patients terminated early from the trial compared to 23.1% of the rest of the 69 patients (χ²=3.2, p=0.07). The effect size for change in suicidal risk measures by S-STS was 0.35 between these 11 patients and the 40 patients assigned citalopram alone.

In comparison, among the two subgroups of citalopram alone and citalopram with inadequate serum lithium levels, the dropout rate was 23% (16/69) compared to 0% (0/11) among the subgroup of patients who had adequate serum lithium levels in addition to taking citalopram. Thus, the pattern of changes in the mean total S-STS was less favorable among these 64 completers, (5.4±5.3 for patients with citalopram alone, 5.1±4.5 for patients with citalopram and inadequate serum lithium levels and 6.7±5.8 for patients with citalopram and adequate serum lithium levels), though higher for those receiving lithium than just citalopram, than in those who received citalopram and had adequate serum lithium levels.

The magnitude of reduction in suicidal thought and behaviors was early in onset and of a much larger magnitude than the reduction of depressive symptoms both by clinical evaluations and patient self-assessment. This finding suggests that suicidal thoughts and behaviors and depressive symptoms are not necessarily linked.

Although the foregoing invention has been described in detail by way of example for purposes of clarity of understanding, it will be apparent to the artisan that certain changes and modifications may be practiced within the scope of the appended claims which are presented by way of illustration not limitation. In this context it will be understood that this invention is not limited to the particular formulations, process steps, and materials disclosed herein as such formulations, process steps, and materials may vary somewhat. It will also be understood that the terminology employed herein is used for the purpose of describing particular embodiments only, and is not intended to be limiting since the scope of the present invention will be limited only by the appended claims and equivalents thereof. It is further noted that various publications and other reference information have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

Ahrens, B., B. Muller-Oerlinghausen. Does Lithium Exert An Independent Antisuicidal Effect? Pharmacopsychiatry (2001), Vol. 34, No. 4, pp. 132-136), Abstract.
American Psychiatric Association. Diagnostic and Statistical Manual of Mental Disorders, 4^{th} Edition. Washington, D.C. 1994.
American Psychiatric Association. Diagnostic and Statistical Manual of Mental Disorders, 4^{th} Edition, Text Revision. Washington, D.C. 2000.
Amorim, P., Y. Lecruiber, E. Weiller, T. Hergueta, D. Sheehan. DSM-III-R Psychotic Disorders: Procedure Validity of The Mini International Neuropsychiatric Interview (M.I.N.I). Concordance And Causes For Discordances With The CIDI. European Psychiatry. 1998, Vol. 13, pp. 26-34.
Andrade L, Caraveo-Anduaga J J, Berglund P, Bijl R V, De Graaf R, Vollebergh W, Dragomirecka E, Kohn R, Keller M, Kessler R C, Kawakami N, Kiliç C, Offord D, Ustun T B, Wittchen H U. The epidemiology of major depressive episodes: results from the International Consortium of Psychiatric Epidemiology (ICPE) Surveys. Int J Methods Psychiatr Res. 2003; 12(1):3-21. Erratum in: Int J Methods Psychiatr Res. 2003; 12(3):165.
Avery D, Winokur G. Mortality in depressed patients treated with electroconvulsive therapy and antidepressants. Archives of general psychiatry. 1976; 33(9):1029-1037.
Baldessarini R J, Tondo L, Hennen J. Lithium treatment and suicide risk in major affective disorders: update and new findings. J Clin Psychiatry. 2003; 64 Suppl 5:44-52.
Baldessarini, R., L. Tondo, and J. Hennen. Treating The Suicidal Patient With Bipolar Disorder: Reducing Suicide Risk With Lithium. Annals of The New York Academy of Sciences. 2001. Vol. 923.
Baldessarini, Ross J., Leonardo Tondo, John Hennen. Effects of Lithium Treatment And Its Discontinuation On Suicidal Behavior In Bipolar Manic-Depressive Disorders. J. Clin. Psychiatry 1999. Vol. 60 (suppl. 2), pp. 77-84.
Barraclough, B., J. Bunch, B. Nelson, and P. Sainsbury. A Hundred Cases of Suicide: Clinical Aspects. The British Journal of Psychiatry. 1974. Vol. 125, pp. 355-373.
Beck, A. T., R. A Steer (1988) Manual for Beck Hopelessness Scale. Psychological Corp., Harcourt Brace Jovanovich San Antonio, Tex.
Beck, A. T., R. A Steer (1991) Manual for Beck Scale For Suicide Ideation. San Antonio, Tex.: Psychological Corporation.
Bleiweiss, et al, U.S. Pat. No. 5,837,701 Composition And Method For Treating Conditions Associated With Symptoms Of Unspecified Retarded Maturation (Issued Nov. 17, 1998).
Boekholdt S M, Peters R J. Rimonabant: obituary for a wonder drug. Lancet. 2010; 376(9740):489-490.
Cade, J. Lithium Sales In The Treatment of Psychotic Excitement. Medical Journal of Australia. 1949, Vol. 36, pp. 349-352.
Center for Disease Control And Prevention. http://www.cdc.gov/ViolencePrevention/suicide/index.html.
Centers for Disease Control and Prevention, National Center for Injury Prevention and Control. Web-based Injury Statistics Query and Reporting System (WISQARS)
Cipriani, A., H. Pretty, K. Hawton, and J. R. Geddes. Lithium In The Prevention of Suicidal Behavior And All-Cause Mortality In Patients With Mood Disorders: A Systematic Review of Randomized Trials. American Journal of Psychiatry. October 2005. Vol. 162, pp. 1805-1819.
Coric, Vladimir, Stock, Elyse G, Pultz, Joseph, Marcus, Ronald, Sheehan, David V. Sheehan Suicidality Tracking Scale (Sheehan-STS): Preliminary Results from a Multicenter Clinical Trial in Generalized Anxiety Disorder. Psychiatry (Edgmont (Pa.: Township)) 2009 6 (1): 26-31.
Dante, Lee, G., U.S. Pat. No. 6,034,091 Method For Treating Emotional Or Mental Illness And Emotional Or Mental Illness Concomitant With Seizures (Issued Mar. 7, 2000).
Fava, G. A., Grandi, M. Zielezny, R. Canestrari, M. A. Morphy. Cognitive Behavioral Treatment of Residual Symptoms In Primary Major Depressive Disorder. Am J. Psychiatry (1995), Vol. 151, No. 9, pp. 1372-1374.
Food and Drug Administration. FDA proposes new warning about suicidal thinking, behavior in young adults who take antidepressant medications. http://www.fda.gov/NewsEvents/Newsroom/PressAnnouncements/2007/ucm108905.htm. Accessed January 2011.
Food and Drug Administration. FDA Requires New Boxed Warnings for the Smoking Cessation Drugs Chantix and Zyban. http://www.fda.gov/Drugs/DrugSafety/DrugSafetyPodcasts/ucm170906.htm. Accessed January 2011.
Food and Drug Administration. FDA Requires Warnings About Risk of Suicidal Thoughts and Behavior for Antiepileptic Medications. http://www.fda.gov/NewsEvents/Newsroom/PressAnnouncements/2008/ucm116991.htm. Accessed January 2011.
Gennaro, A.—Editor. Remington's Pharmaceutical Sciences (17th Ed. 1985), pp. 737-738, 1782, 1788-1790.
Gibbons, R. D., K. Hur, D. K. Bhaumik, and J. J. Mann. The Relationship Between Anti-depressant Prescription Rates And Rates of Early Adolescent Suicide. American Journal of Psychiatry. November 2006, 163:11, pp. 1898-1904.
Gibbons, R. D., K. Hur, S. M. Marcus, D. K. Bhaumik, J. A. Erkens, R. M. C. Herings, and J. J. Mann. Early Evidence On The Effects of Regulators' Suicidality Warnings On SSRI Prescriptions And Suicide In Children And Adolescents. American Journal of Psychiatry. September 2007, 164:9.
Goodman W K, Price L H, Rasmussen S A, et al.: The Yale-Brown Obsessive Compulsive Scale, I: development, use, and reliability. Archives of General Psychiatry 46:1006-1011, 1989a.
Goodwin, F. K., B. Fireman, G. Simon, et al., Suicide Risk In Bipolar Disorder During Treatment With Lithium And Divalproex. Journal of American Medical Association. 2003. Vol. 290:11, pp. 1467-1473.
Gotlib, I. H. & Hammen, C. L. (Eds.). (2002). Handbook of depression. New York: Guilford Press.
Grunebaum, M. F., S. P. Ellis, S. Li, M. A. Oquendo, and J. J. Mann. Anti-depressants And Suicide Risk In The United States, 1985-1999. Journal of Clinical Psychiatry. November 2004. Vol. 65:11, pp. 1456-1462.
Gunell D. Frankel S. Prevention of Suicide: Aspirations And Evidence. BMJ 1994; 308: 1227-1233.
Guy W, ed ECDUE Assessment Manual for Psychopharmacology. Reocville, Md.: US Department of Health, Education and Welfare; 1976.
Guy, W. Editor. Clinical Global Impression (CGI). ECDUE Assessment Manual for Psychopharmacology. 1976. Rockville, Md., US Department of Health, Education and Welfare.
Guzetta, F., L. Tondo, F. Centorrino, and R. J. Baldessarini. Lithium Treatment Reduces Suicide Risk In The United States, 1985-1999. Journal of Clinical Psychiatry. March 2007. Vol. 68, pp. 380-383.
Haahr M. Random.org. http://www.random.org/, Accessed February 2010.
Hamilton M. A rating scale for depression. J. Neurol. Neurosurg. Psychiat., 1960, 23, 56.
Hamilton M: The assessment of anxiety states by rating. British Journal of Medical Psychology 32:50-55, 1959.
Healy, D., C. Langmaak, M. Savage. Suicide In The Course of The Treatment of Depression. J. Psychopharmacol. 1999; 13:94-99
Jamison, Kay R., Ross J. Baldessarini, Effects Of Medical Interventions On Suicidal Behavior, J. Clin. Psychiatry 1999. Vol. 60 (Suppl. 2), pp. 4-6.
Kay S R, Fiszbein A, Opler L A: The Positive and Negative Syndrome Scale (PANSS) for schizophrenia. Schizophrenia Bulletin 13:261-276, 1987.
Kessler R C, Chiu W T, Demler O, Walters E E. Prevalence, severity, and comorbidity of twelve-month DSM-IV disorders in the National Comorbidity Survey Replication (NCS-R). Archives of General Psychiatry, 2005 June; 62(6):617-27.
Khan A, Schwartz K. Suicide risk and symptom reduction in patients assigned to placebo in duloxetine and escitalopram clinical trials: analysis of the FDA summary basis of approval reports. Ann Clin Psychiatry. 2007; 19(1):31-36.
Khan, A., H. Warner, and W. A. Brown. Symptom Reduction And Suicide Risk In Patients Treated With Placebo In Anti-depressant Clinical Trials: An Analysis of The Food And Drug Administration Database. Archives of General Psychiatry. Vol. 57, pp. 311-317, 2000.
Khan, A., S. Khan, R. Leventhal, and W. A. Brown. Suicide Risk In Patients With Anxiety Disorders: A Meta-Analysis of The FDA Database. Journal of Affective Disorders. April 2002. Vol. 68, pp. 183-190.
Khan, A., S. Khan, R. Leventhal, and W. A. Brown. Symptom Reduction And Suicide Risk In Patients Treated With Placebo In Anti-depressant Clinical Trials: A Replication Analysis Of The FDA Database. International Journal of Neuropsychopharmacology. 2001. Vol. 4, pp. 113-118.
Khan, A., S. Khan, R. Leventhal, and W. A. Brown. Symptom Reduction And Suicide Risk Among Patients Treated With Placebo In Antipsychotic Clinical Trials: An Analysis Of The FDA Database. American Journal of Psychiatry. 2001. Vol. 158:9, pp. 1449-1454.
Khan, Arif, Russell L. Kolts, Amy E. Brodhead, K. Ranga Krishnan, Walter A. Brown. Suicide Risk Analysis Among Patients Assigned To Psychotropics And Placebo. Psychopharmacoloty Bulletin 2006. Vol. 39(1), pp. 6-14.
Khan, Arif, Shirin Khan, Russell Kolts, Walter A. Brown, Suicide Rates In Clinical Trials Of SSRIs, Other Anti-depressants, And Placebo: Analysis of FDA Reports. Am. J. Psychiatry 2003. Vol. 160, pp. 790-792.
Leard-Hansson, J., L. Guttmacher. Evidence-Based Psychiatric Medicine—Lithium For Suicide Prevention. Clinical Psychiatry News, August 2009, p. 146.
Lecrubier, Y., D. Sheehan, E. Weiller, P. Amorim, I. Bonara, K. Sheehan, J. Janacs, G. Dunbar. The MINI International Neuropsychiatric Interview (M.I.N.I.). A Short Diagnostic Structured Interview: Reliability And Validity According To The CIDI. European Psychiatry. 1997, Vol. 12, pp. 224-231.
Libby, A. M., H. D. Orton, and R. J. Valuck. Persisting Decline In Treatment After FDA Warnings. Archives of General Psychiatry. June 2009. Vol. 66:6, pp. 633-639.
Liebowitz Social Anxiety Scale Liebowitz M R. Social Phobia. Mod Probl Pharmacopsychiatry 1987; 22:141-173.
Linehan M M, Comtois K A, Murray A M, et al. Two-year randomized controlled trial and follow-up of dialectical behavior therapy vs therapy by experts for suicidal behaviors and borderline personality disorder. Arch Gen Psychiatry. 2006; 63(7):757-766.
Linehan M M. Suicide intervention research: a field in desperate need of development. Suicide Life Threat Behav. 2008; 38(5):483-485.
Mahli, G. S., P. B. Mitchell, S. Salim. Bipolar Depression: Management Options. CNS Drugs (2003), Vol. 17, No. 1, pp. 9-25, Abstract.
Mann J J, Apter A, Bertolote J, et al. Suicide prevention strategies: a systematic review. JAMA. 26 2005; 294(16):2064-2074.
Mann, J. John, Emslie Graham, Ross J. Baldessarini, William Beardslee, Jan A. Fawcett, Frederick K. Goodwin, Andrew C. Leon, Herbert Y. Meltzer, Neal D. Ryan, David Shaffer, Karen D. Wagner. ANCP Task Force Report On SSRIs And Suicidal Behavior In Youth. Neuropsychopharmacology 2006. Vol. 31, pp. 473-492.
Meltzer, H. Y., L. Alps, A. L. Green, C. Altamure, R. Anand, A. Bertoldi, M. Bourgeois, G. Chouinard, M. Z. Islam, J. Kane, R. Krishnan, J. P. Lindenmayer, and S. Potkin. Clozapine Treatment For Treatment of Suicidality In Schizophrenia. Archives of General Psychiatry. January 2003. Vol. 60, pp. 82-91.
Montgomery, S. A. & Åsberg, M. (1979) A New Depression Scale Designed To Be Sensitive To Change. British Journal of Psychiatry. Vol. 134, pp. 382-389.
Muller-Oerlinghausen, B., A. Berghofer. Anti-depressants And Suicidal Risk. Journal of Clinical Psychiatry (1999), Vol. 60 (Suppl. 2), pp. 94-99), Abstract.
Nilsson A. 1999. Lithium Therapy And Suicide Risk. Journal of Clinical Psychiatry. Vol. 60 (Suppl. 2), pp. 85-88.
Ohgami, H., T. Terao, I. Shiotsuki, N. Ishii, and N. Isata. Lithium Levels In Drinking Water And Risk of Suicide. The British Journal of Psychiatry. 2009. Vol. 194, pp. 464-465.
Patorno E, Bohn R L, Wahl P M, et al. Anticonvulsant medications and the risk of suicide, attempted suicide, or violent death. JAMA. 2010; 303(14):1401-1409.
Plata-Salaman, et al., U.S. Pat. No. 7,627,653 Anticonvulsant Derivatives Useful For The Treatment of Depression (Issued Sep. 30, 2003).
Posner K, Oquendo M, Gould M, et al. Columbia Classification Algorithm of Suicide Assessment (C CASA): classification of suicidal events in the FDA's pediatric suicidal risk analysis of antidepressants. Am J Psychiatry. 2007; 164:1035-1043.
Powell, et al., U.S. Pat. No. 4,264,573 Pharmaceutical Formulation For Slow Release Via Controlled Surface Erosion (Issued Apr. 28, 1981).
Prien. R. F., C. J. Klett, and E. M. Caffey. Lithium Prophylaxis In Recurrent Affective Illness. American Journal of Psychiatry. February 1974. Vol. 131, pp. 198-203.
Schrauzer, G. N., and K. P. Shretha. Lithium In Drinking Water And the Incidences of Crimes, Suicides And Arrests Related To Drug Addictions. Biology of Trace Elements of Research. 1990. Vol. 25, pp. 105-113.
Sheehan D. V., Y. Lecrubier, K. Harnett-Sheehan, J. Janacs, E. Weiller, L. I. Bonara, A. Keskiner, J. Schinka, E. Knapp, M. F. Sheehan, G. C. Dunbar. Reliability And Validity of The MINI International Neuropsychiatric Interview (M.I.N.I.): According to the SCID-P. European Psychiatry. 1997, Vol. 12, pp. 232-241.
Sheehan, D. V., Y. Lecrubier, K. Harnett-Sheehan, P. Amorim, J. Janavs, E. Eiller, T. Hergueta, R. Baker, G. Dunbar. The Mini International Neuropsychiatric Interview (M.I.N.I.): The Development and Validation Of A Structured Diatnostic Psychiatric Interview. J. Clin. Psychiatry, 1998, Vol. 59 (Supp. 20), pp. 22-33.
Stoll, Andrew, United States Publication No. US-2003/0012827 Omega-3 Fatty Acids In The Treatment of Depression (Issued Jan. 16, 2003).
Thompson Reuters. Physicians' Desk Reference. 63 ed. Montvale: Thompson Reuters; 2009.
Tollefson, Gary Dennis, United States Publication No. US-2003/0027817 Combination Therapy For Treatment of Bipolar Disorders (Publication: Feb. 6, 2003).
Tondo L, Hennen J, Baldessarini R J. Lower suicide risk with long-term lithium treatment in major affective illness: a meta-analysis. Acta Psychiatr Scand. 2001; 104(3):163-172.
Valuck, R. J., A. M. Libby, J. D. Orton, E. H. Marrato, R. Allen, and R. J. Baldessarini. Spillover Effects On Treatment of Adult Depression In Primary Care After FDA Advisor On Risk Of Pediatric Suicidality With SSRIs. American Journal of Psychiatry. August 2007. Vol. 164:8, pp. 11989-1205.
Walter, G., B. Lyndon, R. Kubb. Lithium AUgmentatioN of Venlafaxine In Adolescent Major Depression. Australian and New Zealand Journal of Psychiatry (1998) Vol. 32, No. 3, pp. 457-459, Abstract.
Weathers, F. W., Ruscio, A. M., & Keane, T. M. (1999). Psychometric properties of nine scoring rules for the Clinician-Administered Posttraumatic Stress Disorder Scale. Psychological Assessment, 11(2), 124-133.
Young R C, Biggs J T, Ziegler V E, Meyer D A. A rating scale for mania: reliability, validity and sensitivity. Br J Psychiatry. 1978; 133:429-435.
Zimmerman M, Mattia J I, Posternak M A. Are subjects in pharmacological treatment trials of depression representative of patients in routine clinical practice? Am J Psychiatry. 2002; 159(3):469-473.

We claim:

1. A method for reducing suicidality in a human subject suffering from a central nervous system (CNS) disorder comprising coordinately administering a CNS therapeutic agent in an amount effective to treat said CNS disorder, and lithium in an amount effective to reduce suicidality in said subject.

2. The method of claim 1, wherein said CNS disorder is selected from a mood disorder, psychosis, or other mental illness

3. The method of claim 1, wherein said CNS disorder is a depressive disorder

4. The method of claim 1, wherein said depressive disorder is selected from mild depression (dysthymia), major depressive disorder, depressive disorder not otherwise specified (NOS), seasonal affective disorder, bipolar disorder, cyclothymic disorder, neurotic depression, atypical (reactive) depression, mood disorders induced by alcohol or drugs, and other mood disorders.

5. The method of claim 4, wherein said depressive disorder is a major depressive disorder (single incidence or recurrent, unipolar depression).

6. The method of claim 1, wherein said CNS disorder is a bipolar or manic depressive disorder

7. The method of claim 1, wherein said CNS disorder is bipolar I disorder or bipolar II disorder.

8. The method of claim 1, wherein said CNS disorder is an anxiety disorder

9. The method of claim 8, wherein said anxiety disorder is selected from generalized anxiety disorder, anxiety disorder not otherwise specified (NOS), obsessive-compulsive disorder (OCD), stress disorders including post-traumatic stress disorder (PTSD), phobias, panic disorders, and anxiety disorders induced by alcohol or drugs.

10. The method of claim 1, wherein said CNS disorder is an appetite or eating disorder.

11. The method of claim 10, wherein said appetite disorder is selected from bulimia nervosa, anorexia nervosa, obesity, and eating disorders not otherwise specified.

12. The method of claim 1, wherein said CNS disorder is an addictive disorder.

13. The method of claim 12, wherein said addictive disorder is selected from alcohol-related addictive disorders, nicotine-related addictive disorders, amphetamine-related addictive disorders, cannabis-related addictive disorders, cocaine-related addictive disorders, hallucinogen use disorders, inhalant-related addictive disorders, and opioid-related addictive disorders.

14. The method of claim 1, wherein said CNS disorder is selected from psychotic disorders, Alzheimer's disease, Parkinson's disease, epilepsy, seizure disorders, tic disorders, attention deficit/hyperactivity disorders, learning disorders, impulse control disorders, oppositional defiant disorders, conduct disorders, abnormal sexual behaviors, schizoid behaviors, somatization, sleep disorders, autism spectrum disorders, and personality disorders.

15. The method of claim 14, wherein said psychotic disorder is selected from schizophrenia, schizophreniform diseases, acute mania, schizoaffective disorders, and depression with psychotic features.

16. The method of claim 1, wherein said CNS therapeutic agent is selected from anti-depressant, anxiolytic, anticonvulsant, antipsychotic, anti-addictive, and appetite suppressant drugs.

17. The method of claim 1, wherein the CNS therapeutic agent is an anti-depressant drug.

18. The method of claim 17, wherein the anti-depressant drug is selected from tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), and indeterminate (atypical) anti-depressants.

19. The method of claim 17, wherein the anti-depressant drug is an SSRI.

20. The method of claim 19, wherein the SSRI is selected from citalopram, excitalopram oxalate, fluvoxamine, paroxetine, fluoxetine, and sertraline.

21. The method of claim 1, wherein the CNS therapeutic agent is citalopram.

22. The method of claim 17, wherein the anti-depressant agent is a tricyclic anti-depressant (TCA).

23. The method of claim 22, wherein the TCA is selected from doxepin, clomipramine, amitriptyline, amitriptyline, maprotiline, desipramine, nortryptyline, desipramine, doxepin, trimipramine, imipramine, and protriptyline.

24. The method of claim 17, wherein the anti-depressant drug is a monoamine oxidase inhibitor (MAOI).

25. The method of claim 22, wherein the MAOI is selected from phenelzine, nortriptyline, selegiline and tranylcypromine.

26. The method of claim 1, wherein the CNS therapeutic agent is administered by a mode of delivery selected from oral, buccal, nasal, aerosol, topical, transdermal, mucosal, or injectable delivery.

27. The method of claim 26, Wherein the CNS therapeutic agent is delivered orally.

28. The method of claim 26, Wherein the CNS therapeutic agent is administered intravenously.

29. The method of claim 1, wherein the CNS therapeutic agent is administered in a sustained release formulation.

30. The method of claim 29, wherein the sustained release formulation provides therapeutically effective plasma levels of said CNS therapeutic agent over a sustained delivery period of approximately 8 hours or longer.

31. The method of claim 29, wherein the sustained release formulation provides therapeutically effective plasma levels of said CNS therapeutic agent over a sustained delivery period of approximately 18 hours or longer.

32. The method of claim 29, wherein the sustained release formulation is a sustained release, oral, transdermal, or injectable formulation.

33. The method of claim 29, wherein the sustained release formulation comprises the CNS therapeutic agent combined with any one or combination of a drug-releasing polymer, matrix, bead, microcapsule, or other solid drug-releasing vehicle, a drug-releasing tiny timed-release pill or mini-tablet; a compressed solid drug delivery vehicle; a controlled release binder; a multi-layer tablet or other multi-layer or multi-component dosage form; a drug-releasing lipid; and a drug-releasing wax.

34. The method of claim 29, wherein the CNS therapeutic agent is administered in a dosage of from about 60 mg to about 1000 mg.

35. The method of claim 29, wherein the CNS therapeutic agent is administered in a once daily dosage form effective to alleviate said CNS disorder.

36. The method of claim 1, wherein the CNS therapeutic agent is citalopram administered in an oral dosage of from about 20 mg to about 1000 mg effective to treat a depressive disorder in said subject.

37. The method of claim 1, wherein the CNS therapeutic agent is citalopram administered in a sustained release formulation.

38. The method of claim 1, wherein the CNS therapeutic agent is an anxiolytic drug.

39. The method of claim 1, wherein the CNS therapeutic agent is an anticonvulsant drug.

40. The method of claim 1, wherein the CNS therapeutic agent is an addiction suppressive drug.

41. The method of claim 1, wherein the CNS therapeutic agent is an appetite suppressant drug.

42. The method of claim 1, wherein said lithium is administered in the form of a lithium salt.

43. The method of claim 42, wherein said lithium salt is selected from the group consisting of citrate, carbonate, chloride, bromide, iodide, sulphate, hydrogen sulphate, nitrate, phosphate, hydrogen phosphate, acetate, lactate, succinate, tartrate, maleate, fumarate, mandelate, acetate, dichloroacetate, trifluoroacetate, oxalate, formate, sulfonate, arginate, asparginate, glutamate, tartrate, and gluconate salts of lithium.

44. The method of claim 1, wherein said lithium is administered in an anti-suicidal effective dosage of from about 50 mg to about 2000 mg.

45. The method of claim 1, wherein said lithium is administered in an anti-suicidal effective dosage of from about 100 mg to about 500 mg.

46. The method of claim 1, wherein said lithium is administered in an anti-suicidal effective dosage of about 500 mg or less.

47. The method of claim 1, wherein said lithium is administered in an anti-suicidal effective dosage of about 300 mg or less.

48. The method of claim 1, wherein said lithium is administered in a sustained release formulation.

49. The method of claim 48, wherein the sustained release formulation is a sustained release, oral, transdermal, or injectable formulation.

50. The method of claim 48, wherein the sustained release formulation comprises lithium combined with any one or combination of a drug-releasing polymer, matrix, bead, microcapsule, or other solid drug-releasing vehicle; a drug-releasing tiny timed-release pill or mini-tablet; a compressed solid drug delivery vehicle; a controlled release binder; a multi-layer tablet or other multi-layer or multi-component dosage form; a drug-releasing lipid; and a drug-releasing wax.

51. The method of claim 1, wherein said CNS therapeutic agent and said lithium are administered to said subject simultaneously

52. The method of claim 1, wherein said CNS therapeutic agent and said lithium are administered in a single, combined dosage form.

53. The method of claim 1, wherein said CNS therapeutic agent and said lithium are admixed or co-formulated in a liquid or solid dosage form.

54. The method of claim 1, wherein said CNS therapeutic agent and said lithium are admixed or co-formulated in an oral dosage form.

55. The method of claim 1, wherein said CNS therapeutic agent and said lithium are administered to said subject at different times during a coordinate dosing period.

56. A pharmaceutical composition for reducing suicidality in a human subject suffering from a central nervous system (CNS) disorder comprising a CNS therapeutic agent in an amount effective to treat said CNS disorder, and lithium in an amount effective to reduce suicidality in said subject, wherein said CNS therapeutic agent and said lithium are admixed or co-formulated in a single, combined dosage form.

57. A pharmaceutical composition according to claim 56, wherein said CNS drug and said lithium are admixed or co-formulated in a liquid or solid dosage form.

58. A pharmaceutical composition according to claim 56, wherein said CNS drug and said lithium are admixed or co-formulated in an oral dosage form further comprising a package containing said combined dosage form, wherein said package includes a label or insert providing instructions for multiple uses of said combined dosage form for treating said CNS disorder and reducing suicidality in said subject.

59. A pharmaceutical composition according to claim 56, wherein said CNS disorder is selected from a mood disorder, psychosis, or other mental illness.

60. A pharmaceutical composition according to claim 56, wherein said CNS disorder is a depressive disorder.

61. A pharmaceutical composition according to claim 60, wherein said depressive disorder is selected from mild depression (dysthymia), major depressive disorder, depressive disorder not otherwise specified (NOS), seasonal affective disorder, bipolar disorder, cyclothymic disorder, neurotic depression, atypical (reactive) depression, mood disorders induced by alcohol or drugs, other mood disorders, and personality disorders, including Borderline Personality Disorder.

62. A pharmaceutical composition according to claim 61, wherein said depressive disorder is a major depressive disorder, including single incidence or recurrent, unipolar depression.

63. A pharmaceutical composition according to claim 56, wherein said CNS disorder is a bipolar or manic depressive disorder.

64. A pharmaceutical composition according to claim 56, wherein said CNS disorder is bipolar I disorder or bipolar II disorder.

65. A pharmaceutical composition according to claim 56, wherein said CNS disorder is an anxiety disorder;

66. A pharmaceutical composition according to claim 65, wherein said anxiety disorder is selected from generalized anxiety disorder, anxiety disorder not otherwise specified (NOS), obsessive-compulsive disorder (OCD), stress disorders including post-traumatic stress disorder (PTSD), phobias, panic disorders, and anxiety disorders induced by alcohol or drugs.

67. A pharmaceutical composition according to claim 56, wherein said CNS disorder is an appetite or eating disorder.

68. A pharmaceutical composition according to claim 67, wherein said appetite disorder is selected from Bulimia Nervosa, anorexia nervosa, obesity, and eating disorders not otherwise specified (NOS).

69. A pharmaceutical composition according to claim 56, wherein said CNS disorder is an addictive disorder.

70. A pharmaceutical composition according to claim 69, wherein said addictive disorder is selected from alcohol-related addictive disorders, nicotine-related addictive disorders, amphetamine-related addictive disorders, cannabis-related addictive disorders, cocaine-related addictive disorders, hallucinogen use disorders, inhalant-related addictive disorders, and opioid-related addictive disorders.

71. A pharmaceutical composition according to claim 56, wherein said CNS disorder is selected from psychotic disorders, Alzheimer's disease, Parkinson's disease, epilepsy, seizure disorders, tic disorders, attention deficit/hyperactivity disorders, learning disorders, impulse control disorders, oppositional defiant disorders, conduct disorders, abnormal sexual behaviors, schizoid behaviors, somatization, sleep disorders, autism spectrum disorders, and personality disorders.

72. A pharmaceutical composition according to claim 71, wherein said psychotic disorder is selected from schizophrenia, schizophreniform diseases, acute mania, schizoaffective disorders, and depression with psychotic features.

73. A pharmaceutical composition according to claim 56, wherein said CNS therapeutic agent is selected from anti-depressant, anxiolytic, anticonvulsant, antipsychotic, antiaddictive, and appetite suppressant drugs.

74. A pharmaceutical composition according to claim 73, wherein the anti-depressant drug is selected from tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), and indeterminate (atypical) anti-depressants.

75. A pharmaceutical composition according to claim 74, wherein the anti-depressant drug is a selective serotonin reuptake inhibitor (SSRI).

76. A pharmaceutical composition according to claim 75, wherein the SSRI is selected from citalopram, excitalopram oxalate, fluvoxamine, paroxetine, fluoxetine, and sertraline.

77. A pharmaceutical composition according to claim 56, wherein the CNS therapeutic agent is citalopram.

78. A pharmaceutical composition according to claim 74, wherein the anti-depressant drug is a tri-cyclic anti-depressant (TCA).

79. A pharmaceutical composition according to claim 78, wherein the TCA is selected from doxepin, clomipramine, amitriptyline, amitriptyline, maprotiline, desipramine, nortryptyline, desipramine, doxepin, trimipramine, imipramine, and protriptyline.

80. A pharmaceutical composition according to claim 74, wherein the anti-depressant drug is a monoamine oxidase inhibitor (MAOI).

81. A pharmaceutical composition according to claim 80, wherein the MAOI is selected from phenelzine, nortriptyline, selegiline and tranylcypromine.

82. A pharmaceutical composition according to claim 56, wherein the CNS drug is formulated for administration by a mode of delivery selected from selected from oral, buccal, nasal, aerosol, topical, transdermal, mucosal, or injectable delivery.

83. A pharmaceutical composition according to claim 82, wherein the CNS drug is formulated for oral administration.

84. A pharmaceutical composition according to claim 82, wherein the CNS drug is formulated for injectable administration.

85. A pharmaceutical composition according to claim 56, wherein the CNS drug is formulated in an extended release or sustained release formulation.

86. A pharmaceutical composition according to claim 82, wherein the sustained release formulation provides therapeutically effective plasma levels of said CNS drug over a sustained delivery period of approximately 8 hours or longer.

87. A pharmaceutical composition according to claim 82, wherein the sustained release formulation provides therapeutically effective plasma levels of said CNS drug over a sustained delivery period of approximately 18 hours or longer.

88. A pharmaceutical composition according to claim 85, wherein the extended or sustained release formulation is a sustained release, oral, transdermal, or injectable formulation.

89. A pharmaceutical composition according to claim 56, wherein the CNS drug is administered in a dosage of from about 60 mg to about 1000 mg.

90. A pharmaceutical composition according to claim 56, wherein the CNS drug is formulated in a once daily dosage form effective to alleviate said CNS disorder.

91. A pharmaceutical composition according to claim 56, wherein the CNS drug is citalopram administered in an oral dosage effective to treat a depressive disorder in said subject.

92. A pharmaceutical composition according to claim 56, wherein the CNS drug is citalopram administered in a sustained release formulation.

93. A pharmaceutical composition according to claim 56, wherein the CNS therapeutic agent is an anxiolytic drug.

94. A pharmaceutical composition according to claim 56, wherein the CNS therapeutic agent is an anticonvulsant drug.

95. A pharmaceutical composition according to claim 56, wherein the CNS therapeutic agent is an addiction suppressive drug.

96. A pharmaceutical composition according to claim 56, wherein the CNS therapeutic agent is an addiction an appetite suppressant drug.

97. A pharmaceutical composition according to claim 56, wherein said lithium is administered in the form of a lithium salt.

98. A pharmaceutical composition according to claim 97, wherein said lithium salt is selected from the group consisting of citrate, carbonate, chloride, bromide, iodide, sulphate, hydrogen sulphate, nitrate, phosphate, hydrogen phosphate, acetate, lactate, succinate, tartrate, maleate, fumarate, mandelate, acetate, dichloroacetate, trifluoroacetate, oxalate, formate, sulfonate, arginate, asparginate, glutamate, tartrate, and gluconate salts of lithium.

99. A pharmaceutical composition according to claim 56, wherein said lithium is administered in an antisuicidal effective dosage of from about 50 mg to about 2000 mg.

100. A pharmaceutical composition according to claim 56, wherein said lithium is administered in an antisuicidal effective dosage of from about 100 mg to about 500 mg.

101. A pharmaceutical composition according to claim 56, wherein said lithium is administered in an antisuicidal effective dosage of about 500 mg or less.

102. A pharmaceutical composition according to claim 56, wherein said lithium is administered in an antisuicidal effective dosage of about 300 mg or less.

103. A pharmaceutical composition according to claim 56, wherein said lithium is formulated in an extended or sustained release formulation.

104. A pharmaceutical composition according to claim 103, wherein the sustained release formulation is a sustained release, oral, transdermal, or injectable formulation.

105. A pharmaceutical composition according to claim 56, wherein the sustained release formulation comprises lithium combined with any one or combination of a drug-releasing polymer, matrix, bead, microcapsule, or other solid drug-releasing vehicle, a drug-releasing tiny timed-release pill or mini-tablet; a compressed solid drug delivery vehicle; a controlled release binder; a multi-layer tablet or other multi-layer or multi-component dosage form; a drug-releasing lipid; and a drug-releasing wax.

106. A pharmaceutical composition according to claim 56, wherein said CNS drug and said lithium are formulated in a combined dosage form for simultaneous administration.

107. A pharmaceutical composition according to claim 56, wherein said CNS drug and said lithium are admixed or co-formulated in a liquid or solid dosage form.

108. A pharmaceutical composition according to claim 56, wherein said CNS drug and said lithium are admixed or co-formulated in an oral dosage form.

109. A pharmaceutical kit for reducing suicidality in a human subject suffering from a central nervous system (CNS) disorder comprising a CNS therapeutic agent in an amount effective to treat said CNS disorder, lithium in an amount effective to reduce suicidality in said subject, and container means for containing said CNS therapeutic agent and said lithium for coordinate administration to said subject.

110. A pharmaceutical kit according to claim 109, wherein said container means comprises a package including a label or insert providing instructions for multiple uses.

111. A pharmaceutical kit according to claim 109, wherein said kit contains said CNS therapeutic agent and said lithium admixed or co-formulated in a single, combined dosage form.

112. A pharmaceutical kit according to claim 111, wherein said CNS drug and said lithium are admixed or co-formulated in a liquid or solid dosage form.

113. A pharmaceutical kit according to claim 111, wherein said CNS drug and said lithium are admixed or co-formulated in an oral dosage form.

114. A pharmaceutical kit according to claim 111, wherein said CNS therapeutic agent and said lithium are provided in separate dosage forms.

115. A method for reducing suicidality in a human subject at risk for suicide comprising coordinately administering a CNS therapeutic agent and lithium in an amount effective to reduce suicidality in said subject.

116. The method of claim 115, wherein said CNS therapeutic agent is selected from anti-depressant, anxiolytic, anticonvulsant, antipsychotic, anti-addictive, and appetite suppressant drugs.

117. The method of claim 116, wherein the CNS therapeutic agent is an anti-depressant drug.

118. The method of claim 117, wherein the anti-depressant drug is selected from tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), and indeterminate (atypical) anti-depressants.

119. The method of claim 118, wherein the anti-depressant drug is an SSRI.

120. The method of claim 115, wherein the CNS therapeutic agent is citalopram.

121. The method of claim 115, wherein the CNS therapeutic agent is administered by a mode of delivery selected from oral, buccal, nasal, aerosol, topical, transdermal, mucosal, or injectable delivery.

122. The method of claim 115, wherein the CNS therapeutic agent is administered in a sustained release formulation.

123. The method of claim 121, wherein the CNS therapeutic agent is administered in a dosage of from about 60 mg to about 1000 mg.

124. The method of claim 115, wherein said lithium is administered in the form of a lithium salt.

125. The method of claim 124, wherein said lithium salt is selected from the group consisting of citrate, carbonate, chloride, bromide, iodide, sulphate, hydrogen sulphate, nitrate, phosphate, hydrogen phosphate, acetate, lactate, succinate, tartrate, maleate, fumarate, mandelate, acetate, dichloroacetate, trifluoroacetate, oxalate, formate, sulfonate, arginate, asparginate, glutamate, tartrate, and gluconate salts of lithium.

126. The method of claim 115, wherein said lithium is administered in an anti-suicidal effective dosage of from about 50 mg to about 2000 mg.

127. The method of claim 115, wherein said lithium is administered in an anti-suicidal effective dosage of from about 100 mg to about 500 mg.

128. The method of claim 115, wherein said lithium is administered in an anti-suicidal effective dosage of about 500 mg or less.

129. The method of claim 115, wherein said lithium is administered in an anti-suicidal effective dosage of about 300 mg or less.

130. The method of claim 115, wherein said lithium is administered in a sustained release formulation.

131. The method of claim 115, wherein said CNS therapeutic agent and said lithium are administered to said subject simultaneously

132. The method of claim 115, wherein said CNS therapeutic agent and said lithium are administered in a single, combined dosage form.

133. The method of claim 115, wherein said CNS therapeutic agent and said lithium are admixed or co-formulated in a liquid or solid dosage form.

134. The method of claim 115, wherein said CNS therapeutic agent and said lithium are admixed or co-formulated in an oral dosage form.

135. The method of claim 115, wherein said CNS therapeutic agent and said lithium are administered to said subject at different times during a coordinate dosing period.

136. A pharmaceutical composition for reducing suicidality in a human subject at risk for suicide comprising a CNS therapeutic agent and lithium in an amount effective to reduce suicidality in said subject, wherein said CNS therapeutic agent and said lithium are admixed or co-formulated in a single, combined dosage form.

137. A pharmaceutical composition according to claim 136, wherein said CNS drug and said lithium are admixed or co-formulated in a liquid or solid dosage form.

138. A pharmaceutical composition according to claim 136, wherein said CNS drug and said lithium are admixed or co-formulated in an oral dosage form further comprising a package containing said combined dosage form, wherein said package includes a label or insert providing instructions for multiple uses of said combined dosage form for treating said CNS disorder and reducing suicidality in said subject.

139. The pharmaceutical composition according to claim 136, wherein said CNS therapeutic agent is selected from anti-depressant, anxiolytic, anticonvulsant, antipsychotic, antiaddictive, and appetite suppressant drugs.

140. The pharmaceutical composition according to claim 139, wherein the anti-depressant drug is selected from tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), and indeterminate (atypical) anti-depressants.

141. The pharmaceutical composition according to claim 140, wherein the anti-depressant drug is a selective serotonin reuptake inhibitor (SSRI).

142. The pharmaceutical composition according to claim 136, wherein the CNS therapeutic agent is citalopram.

143. The pharmaceutical composition according to claim 136, wherein the CNS drug is formulated for administration by a mode of delivery selected from selected from oral, buccal, nasal, aerosol, topical, transdermal, mucosal, or injectable delivery.

144. The pharmaceutical composition according to claim 136, wherein the CNS drug is formulated in an extended release or sustained release formulation.

145. The pharmaceutical composition according to claim 136, wherein the CNS drug is administered in a dosage of from about 60 mg to about 1000 mg.

146. The pharmaceutical composition according to claim 136, wherein said lithium is administered in the form of a lithium salt.

147. The pharmaceutical composition according to claim 146, wherein said lithium salt is selected from the group consisting of citrate, carbonate, chloride, bromide, iodide, sulphate, hydrogen sulphate, nitrate, phosphate, hydrogen phosphate, acetate, lactate, succinate, tartrate, maleate, fumarate, mandelate, acetate, dichloroacetate, trifluoroacetate, oxalate, formate, sulfonate, arginate, asparginate, glutamate, tartrate, and gluconate salts of lithium.

148. The pharmaceutical composition according to claim 147, wherein said lithium is administered in an antisuicidal effective dosage of from about 50 mg to about 2000 mg.

149. The pharmaceutical composition according to claim 136, wherein said lithium is administered in an antisuicidal effective dosage of from about 100 mg to about 500 mg.

150. The pharmaceutical composition according to claim 136, wherein said lithium is administered in an antisuicidal effective dosage of about 500 mg or less.

151. The pharmaceutical composition according to claim 136, wherein said lithium is administered in an antisuicidal effective dosage of about 300 mg or less.

152. The pharmaceutical composition according to claim 136, wherein said lithium is formulated in an extended or sustained release formulation.
